(19) United States
(12) Patent Application Publication
(10) Pub. No.: US 2016/0136192 A1
(43) Pub. Date: May 19, 2016

(54) METHODS AND COMPOSITIONS FOR ENHANCING COGNITIVE PERFORMANCE

(71) Applicant: ABBOTT LABORATORIES, Abbott Park, IL (US)

(72) Inventors: REMA VAZHAPPILLY, Singapore (SG); TAPAS DAS, Worthington, OH (US)

(73) Assignee: ABBOTT LABORATORIES, Abbott Park, IL (US)

(21) Appl. No.: 14/897,462

(22) PCT Filed: Jun. 10, 2014
(86) PCT No.: PCT/US14/41630

§ 371 Date: Dec. 10, 2015

Related U.S. Application Data
(60) Provisional application No. 61/833,389, filed on Jun. 10, 2013.

(51) Int. Cl. A61K 31/685; A61K 31/201; A61K 9/00; A61K 31/14

(52) U.S. Cl. CPC A61K 31/685; A61K 31/14; A61K 31/201; A61K 9/0095; A61K 9/0056; A23L 1/296; A23V 2002/00

(57) ABSTRACT

Compositions including an effective amount of each of phosphatidyl serine, choline, and oleic acid, and methods for improving cognitive performance that include administering such compositions to a subject, are described herein. The compositions can be nutritional compositions such as bars or liquids suitable for oral administration. The combination of phosphatidyl serine, choline, and oleic acid provides synergistic or complimentary modes of action that improve the overall effect of these compounds.

### CROSS REFERENCE TO RELATED APPLICATIONS

This application claims priority to and the benefit of U.S. Application No. 61/833,389, filed Jun. 10, 2013, the entire content of which is incorporated herein by reference.

### BACKGROUND

The elderly often experience declines in memory and cognition, and this is borne out from longitudinal studies of cognitive aging. Zelinski et al., Psychol. Aging, 13, 622-30 (1998). Poor health, decreased physical activity, high blood pressure, medications, and vitamin deficiencies affect memory negatively, whereas good health, increased physical and mental activity, a higher level of education, and decreased depression all have positive influences on memory. These findings suggest that preventative strategies for sustaining high intellectual performance in later life may be possible. Calvaresi et al., Journal Gerontology: Psychological Sciences, 56(6), P327-P339 (2001). Some of these strategies adapted by people are the use of nutraceuticals.

Nutraceuticals are commonly defined as any substance that is considered a food, a part of a food, a vitamin, a mineral, or an herb that provides health benefits, including disease prevention and/or treatment. They include a new class of product, the dietary supplement, which is considered neither food nor drug and is not subject to the same regulatory hurdles as prescription and over-the-counter medicines. Products in this category include vitamins, minerals, herbs, amino acids, and other substances that are not intended as a substitute for food. With the increased interest in nutraceuticals, medical and allied health care professionals interested in holistic practices, including medical and research scientists, are carrying out clinical trials to determine whether these treatments have any merit or produce the stated results. Sand-Jecklin et al., Journal of Holistic Nursing, 21(4), 383-397 (2003).

The market for nutraceuticals to enhance and maintain memory function is booming. Two national surveys of adults in the United States were reported. In the first study, the researchers found that adults greater than 60 years of age had a higher use of all supplement types than did younger age groups. Radimer et al., American Journal Epidemiology, 160, 339-349 (2004). In the other survey, 65% of older adults reported that they used dietary supplements. Examples include the herb Ginkgo Biloba, which is sold as a memory enhancer, Gotu Kola which is used as a stimulant, and Huperzine, which is a moss-derived alkaloid which has been used in China to treat dementia. However, there are a number of problems associated with existing nutraceuticals used for cognitive decline and memory enhancement. First, some ingredients are completely homeopathic and contain components not known outside of the homeopathic field. Second, the evidence of treatment efficacy is often contradictory. Finally, there is often little or no research demonstrating the effectiveness of the nutraceuticals.

### SUMMARY

A combination of phosphatidyl serine, choline, and oleic acid for use in nutritional compositions is described herein. The combination of effective amounts of each of phosphatidyl serine, choline, and oleic acid has beneficial synergistic and/or complementary modes of action that are effective for improving cognitive performance and brain health.

Provided herein is a method of improving cognitive performance, comprising orally administering a composition comprising an effective amount of each of phosphatidyl serine, choline, and oleic acid to a subject. Also provided herein is a combination comprising phosphatidyl serine, choline, and oleic acid for use in improving the cognitive performance of a human. Further disclosed herein is the use of a combination comprising phosphatidyl serine, choline, and oleic acid in the manufacture of a nutritional composition for improving the cognitive performance of a human.

Also provided herein is a nutritional composition comprising phosphatidyl serine, choline, oleic acid, at least one source of carbohydrate, and at least one source of protein.

### BRIEF DESCRIPTION OF THE DRAWINGS

FIG. 1A illustrates the effect of choline (30 micromolar) on field Excitatory Post Synaptic Potentials (fEPSP) amplitude in hippocampal slices (in vitro) as analyzed in Example 4.

FIG. 1B illustrates the effect of choline (40 micromolar) on fEPSP amplitude in hippocampal slices (in vitro) as analyzed in Example 4.

FIG. 1C illustrates the effect of choline (50 micromolar) on fEPSP amplitude in hippocampal slices (in vitro) as analyzed in Example 4.

FIG. 1D illustrates the effect of choline (60 micromolar) on fEPSP amplitude in hippocampal slices (in vitro) as analyzed in Example 4.

FIG. 1E illustrates the effect of choline (at various concentrations) on the percentage of fEPSP amplitude in hippocampal slices (in vitro) as analyzed in Example 4. The Y-axis represents the % of fEPSP amplitude (compared to control); *=p<0.05; **=p<0.01; ***=p<0.001.

FIG. 2A illustrates the effect of oleic acid (10 micromolar) on fEPSP amplitude in hippocampal slices (in vitro) as analyzed in Example 5.

FIG. 2B illustrates the effect of oleic acid (20 micromolar) on fEPSP amplitude in hippocampal slices (in vitro) as analyzed in Example 5.

FIG. 2C illustrates the effect of oleic acid (30 micromolar) on fEPSP amplitude in hippocampal slices (in vitro) as analyzed in Example 5.

FIG. 2D illustrates the effect of oleic acid (at various concentrations) on the percentage of fEPSP amplitude in hippocampal slices (in vitro) as analyzed in Example 5.

### DETAILED DESCRIPTION

Unless otherwise defined, all technical and scientific terms used herein have the same meaning as commonly understood by one of ordinary skill in the art to which this application pertains. Although methods and materials similar or equivalent to those described herein can be used in the practice or testing of the exemplary embodiments, suitable methods and materials are described below. In case of conflict, the present specification, including definitions, will control. In addition, the materials, methods, and examples are illustrative only and not intended to be limiting.

Provided herein is a method of improving cognitive performance, comprising orally administering a composition comprising an effective amount of each of phosphatidyl serine, choline, and oleic acid to a subject. Also provided herein is a combination comprising phosphatidyl serine, choline, and oleic acid for use in improving the cognitive performance of a human. Further disclosed herein is the use of a combination comprising phosphatidyl serine, choline, and oleic acid in the manufacture of a nutritional composition for improving the cognitive performance of a human.

Also provided herein is a nutritional composition comprising phosphatidyl serine, choline, oleic acid, at least one source of carbohydrate, and at least one source of protein.

### DEFINITIONS

The terminology as set forth herein is for description of the embodiments only and should not be construed as limiting the application as a whole. Unless otherwise specified, “a,” “an,” “the,” and “at least one” are used interchangeably. Furthermore, as used in the description of the application and the appended claims, the singular forms “a”, “an”, and “the” are inclusive of their plural forms, unless contraindicated by the context surrounding such.

The recitations of numerical ranges by endpoints include all numbers subsumed within that range (e.g., 1 to 5 includes 1, 1.5, 2, 2.75, 3, 3.80, 4, 5, etc.).

The expression “effective amount” as used herein, refers to a sufficient amount of active ingredients to enhance cognitive performance such as memory or intelligence. The exact amount required will vary from subject to subject, depending on the species, age, and general condition of the subject and the like.

The term “elderly,” as used herein, refers to an individual of at least 45 years of age, including at least 50 years of age, at least 55 years of age, at least 60 years of age, at least 65 years of age, at least 70 years of age, at least 75 years of age, and including at least 80 years or age or greater. The term “elderly” also includes the groups of from about 45 years of age to about 95 years of age, and the group of from about 55 years of age to about 80 years of age.

The subject can be a mammal, such as a human, a domesticated farm animal (e.g., cow, horse, pig) or pet (e.g., dog, cat). More preferably, the subject is a human. In another embodiment, the subject is a female. In another embodiment, the subject is a male. In another embodiment, the subject is a baby. In another embodiment, the subject is a child. In another embodiment, the subject is a young child. In another embodiment, the subject is an adult. In another embodiment, the subject is an elderly adult.

“Baby” refers, in another embodiment, to a subject under the age of 1 year. “Child” refers, in another embodiment, to a subject under the age of 18 years. “Young child” refers, in another embodiment, to a subject under the age of 7 years. “Adult” refers, in other embodiments, to a subject over 18. An elderly adult is an adult who is 45 years or older. Human subjects can be selected from any age group. For example subjects can have an age from 1 to 100+, or any ages there between.

Compositions comprising an effective amount of each of phosphatidyl serine, choline, and oleic acid, and methods of using such compositions for improving cognitive performance by administering them to a subject are described herein. Use of this combination has been shown to provide a synergistic or complementary effect on cognitive function. For the sake of convenience, the combination of phosphatidyl serine, choline, and oleic acid are also referred to herein as the active ingredients.

### The Active Ingredients

Phosphatidyl serine (PS), which has the chemical name 2-diacyl-sn-glycero-3-phospho-L-serine occurs widely in animals, plants and microorganisms. Since the brain is enriched with PS, there may be a preferential dietary uptake of PS compared to other phospholipids. Based on the results of clinical studies, the cognitive benefits of PS are supported by an FDA qualified health claim which states that “consumption of phosphatidyl serine may reduce the risk of dementia or cognitive dysfunction in the elderly.” Membrane PS has been shown to be required for the activation of phosphatidylinositol 3-kinase (PI3K)/AKT serine/threonine protein kinase (AKT)/mammalian target of rapamycin kinase (mTOR) signaling pathway. The phosphatidylinositol 3-kinase (PI3K)/AKT serine/threonine protein kinase (AKT)/mammalian target of rapamycin kinase (mTOR) signaling pathway serves as a link between aspects of learning and memory, neuronal survival, neurogenesis, and apoptosis. Therefore, PS appears to be involved in modulating the phosphatidylinositol 3-kinase (PI3K)/AKT serine/threonine protein kinase (AKT)/mammalian target of rapamycin kinase (mTOR) signaling pathway, which plays a critical role in learning and memory.

Choline (C), which has the chemical name 2-hydroxy-N,N,N-trimethylethanaminium is a water-soluble essential nutrient that is commonly classified with the B-complex vitamins. Choline is a quaternary ammonium salt in which the positively charged trimethylethanaminium ion is paired with a negative ion such as chloride, hydroxide, or tartrate. The choline cation is found in the head groups of phosphatidylcholine and sphingomyelin, two classes of phospholipid that are abundant in cell membranes. Choline is also the precursor molecule for the neurotransmitter acetylcholine which is involved in many functions including memory and muscle control.

Oleic acid (OA) is a fatty acid that occurs naturally in various animal and vegetable fats and oils. In chemical terms, oleic acid is classified as a monounsaturated omega-9 fatty acid. It has the formula CH₃(CH₂)₇CH═CH(CH₂)₇COOH, and the chemical name (9Z)-Octadec-9-enoic acid. Oleic acid consumption has been shown to lower Low Density Lipoprotein (LDL) levels and increase High Density Lipoprotein (HDL) levels, and therefore has been indicated for use in promoting heart health.

Multiple molecular mechanisms contribute to the pathophysiology of cognitive impairment and brain dysfunction in neurodegenerative diseases. The methods described herein use a combination of phosphatidyl serine, choline, and oleic acid to affect multiple molecular mechanisms, resulting in an additive or synergistic effect in enhancing cognitive performance and brain health.

Phosphatidyl serine, choline, and oleic acid also have a shared effect on certain neurochemical pathways. For example, these compounds may enhance N-methyl-D-aspartate receptor (NMDAR) dependant hippocampal Long Term Potentiation (LTP) that is a measure of synaptic plasticity, an important molecular mechanism involved in cognition. Accordingly, in certain embodiments of the methods and compositions disclosed herein, the administration of an effective amount of each of phosphatidyl serine, choline and oleic acid to a subject results in enhancement of NMDAR dependent hippocampal LTP, thereby resulting in an enhancement in the cognitive performance of the subject. Phosphatidyl serine, choline, and oleic acid may also all effect brain derived neurotrophic factor (BDNF), which is a neurotrophic factor that enhances the effects of voluntary physical activity on synaptic plasticity. Accordingly, in certain embodiments of the methods and compositions disclosed herein, the administration of an effective amount of each of phosphtidyl serine, choline and oleic acid to a subject results in an improvement in BDNF, thereby resulting in an enhancement in the cognitive performance of the subject.

### Cognitive Performance

Cognitive performance can be categorized and tested in a variety of ways. Major categories of cognitive performance include memory and intelligence. However, a wide variety of more complex cognitive functions are known, including verbal memory, implicit memory and learning, and those supported by an individual's ability to organize information, such as executive function.

As used herein, the terms “improving” or “improvement” of cognitive performance refer generally to increasing the memory capacity or intelligence of the subject. In certain embodiments, the terms refer to an increased or improved baseline level of the memory or intelligence in the subject. In other embodiments, the terms refer to an increased or improved level of the memory or intelligence in the subject.

In another embodiment, “improvement” of cognitive performance such as memory or intelligence refers to effecting a 10% (or greater) improvement thereof. In another embodiment, the term refers to effecting a 20% (or greater) improvement in cognitive performance such as memory, intelligence or both. In another embodiment, the term refers to effecting a 30% (or greater) improvement in cognitive performance such as memory, intelligence or both. In another embodiment, the term refers to effecting a 40% (or greater) improvement in cognitive performance such as memory, intelligence or both. In another embodiment, the term refers to effecting a 50% (or greater) improvement in cognitive performance such as memory, intelligence or both. In another embodiment, the term refers to effecting a 60% (or greater) improvement in cognitive performance such as memory, intelligence or both. In another embodiment, the term refers to effecting a 70% (or greater) improvement in cognitive performance such as memory, intelligence or both. In another embodiment, the term refers to effecting an 80% (or greater) improvement in cognitive performance such as memory, intelligence or both. In another embodiment, the term refers to effecting a 90% (or greater) improvement in cognitive performance such as memory, intelligence or both. In another embodiment, the term refers to effecting a 100% (or greater) improvement in cognitive performance such as memory, intelligence or both. It should be understood that when a greater percentage of improvement is referred to (e.g., a 20% improvement in cognitive performance) it necessarily includes lesser amounts of improvement (e.g., a 10% improvement). In other words, a subject achieving a 10% improvement may also (in certain embodiments) achieve a 20% improvement such that the 10% improvement should not necessarily be understood to be limited to an improvement of no more than 10%.

In another embodiment, improvement of a cognitive performance such as memory or intelligence is assessed relative to the cognitive memory or intelligence before beginning treatment. In another embodiment, improvement of a cognitive memory or intelligence is assessed relative to an untreated subject. In another embodiment, improvement of a cognitive performance such as memory or intelligence is assessed according to a standardized criterion such as, for example, a test or the like.

One aspect of intelligence is executive function. Executive function is a term used to describe a number of distinct, specifiable ‘control’ functions that are distinguishable from processing speed, memory and motor functions. Examples of executive functions include ‘switching’ or ‘shifting’ (e.g. alternating between behaviors or information sources), ‘inhibition’ (the ability to suppress automatic and habitual responses or behaviors, ‘updating’ (the ability to discard and replace information, ‘sustained attention’ (requiring sustained concentration and monitoring skills), ‘strategic memory search’ (conscious, controlled retrieval of structured information) and ‘planning’ (the ability to deal with novel information, generate goals and make decisions on a suitable course of action). Rabbitt P., “Methodology of frontal and executive function.” Psychology Press, Hove (1997). All of the above processes are dependent on ‘working memory’, a psychological construct used to describe a hypothetical system for the temporary manipulation and maintenance of speech-based and/or visuospatial information, requiring the control of attentional resources

In terms of memory and learning, various distinctions can be drawn between short-term memory (e.g., digit span or digit recall tasks) and long-term memory (e.g., episodic immediate or delayed recall and recognition), conscious or unconscious processes (e.g., explicit versus implicit forms of memory and learning), memory for events (episodic memory) or meaning (semantic memory), remembering to perform actions (prospective memory), memory for skills (procedural memory) and memory for images and/or spatial orientation (visuospatial or spatial memory). Motor function is measured with or without a cognitive component (e.g., motor speed or psychomotor speed), and IQ may be sub-divided into crystallized intelligence (measuring acquired knowledge) and fluid intelligence (measuring non-verbal ability, problem-solving and pattern recognition independent of acquired knowledge).

The effects of the combination of phosphatidyl serine, choline, and oleic acid on cognitive function should utilize a wide range of tasks for a full assessment. In doing so, it is important to bear in mind two points. First, although a particular task might be identified as having a primary neuropsychological focus such as ‘executive function’ or ‘episodic memory’, such measures are not ‘task pure’. For example, a range of processes may support a nominally ‘executive’ task such as memory, processing speed and motor function. Second, in terms of the underlying brain regions supporting cognitive performance, it is important to recognize that any task is likely to recruit multiple neural regions. For example, functional neuroimaging studies have revealed activations in prefrontal cortex, medial and lateral parietal cortex, as well as hippocampal/medial temporal lobe activations during episodic memory retrieval.

Animal studies can be used to evaluate simple neurochemical or neuroprotective effects of the active ingredients. Cognitive tests for the effect of administration of active ingredients (i.e., PS, C, and OA) on more complex cognitive functions rely primarily on two things: (1) the potential for cognitive change as a result of administration of the active ingredients with respect to dose and duration in the cognitive domain or cognitive aspect being measured and (2) cognitive methodologies sensitive enough to measure such cognitive change. The most important consideration in setting up a suitable framework for measuring human cognitive function is to determine methods that are sensitive to dietary changes and repeatable over time, simple to interpret and specific to cognitive domains. In this respect, brief measures, such as the Mini-Mental Status Examination (MMSE) (Folstein et al., J. Psychiatr. Res. 12, 189-198 (1975)) and the Alzheimer's Disease Assessment Scale Cognitive Subscale (ADAS-Cog) (Rosen et al., Am J. Psychiatry 141, 1356-1364 (1984)), are suitable for cognitive screening of dementia and mild cognitive impairment (MCI), a term generally used to describe the level of cognitive impairment found in the intermediate stage between normal ageing and fully developed dementia, and also for the measurement of widespread, gross cognitive changes over time in longitudinal studies. Both tasks consist of 11 items, covering a broad range of cognitive functions: orientation, attention and calculation, memory, language and motor skills. A discussion of 55 different cognitive tests useful for evaluating the effects of dietary intervention can be helpful in determining which tests should be used. See Macready et al., Genes Nutr. 4, 227-242 (2009), the disclosure of which is incorporated herein by reference.

In some embodiments, the combination of PS, C, and OA are administered to a subject having (including being diagnosed with) one or more of a central nervous system disorder, cognitive deficits and dementias associated with a diversity of conditions, including age-related or glucocorticoid-related declines in cognitive function such as those seen in Alzheimer's and associated dementias, major depressive disorder, psychotic depression, anxiety, panic disorder, post traumatic stress disorder, depression in Cushing's syndrome, and treatment-resistant depression. In a further embodiment, the subject is an elderly human who has been diagnosed with dementia or cognitive dysfunction.

### Formulation and Administration

The active ingredients can be formulated in a suitable composition and then, in accordance with the methods described herein, administered to a subject in a form adapted to the chosen route of administration. The formulations include those suitable for oral administration. Oral administration, as defined herein, includes any form of administration in which the active ingredients passes through the esophagus of the subject. For example, oral administration includes nasogastric intubation, in which a tube is run from through the nose to the stomach of the subject to administer food or drugs.

Pharmaceutical and nutritional formulations (e.g., nutritional compositions) including phosphatidyl serine, choline, and oleic acid can also be referred to herein as medicaments. For example, a combination of phosphatidyl serine, choline, and oleic acid can be used for the preparation of a medicament for treating a subject in need of cognitive improvement. In a further embodiment, the nutritional formulation can be used for a subject that has been diagnosed with dementia or cognitive dysfunction.

Compositions including effective amounts of each of phosphatidyl serine, choline, and oleic acid, such as nutritional compositions, can be provided to a subject in one or more servings over a period of time. The term “serving” as used herein, unless otherwise specified, is intended to be construed as any amount which is intended to be consumed by an individual in one sitting or within one hour or less. In certain embodiments, the nutritional composition is provided or administered to a subject in an amount of two servings per day. In other embodiments, the nutritional composition is provided or administered to a subject in an amount of two or more servings per day.

The active ingredients can be administered to a subject one or more times per day for a period suitable to achieve the desired effect. For example, a composition comprising an effective amount of each of phosphatidyl serine, choline, and oleic acid can be administered to a subject every day for at least a week, every day for at least two weeks, every day for at least a month, every day for at least 6 months, or every day for a year or more. As another example, a composition comprising an effective amount of each of phosphatidyl serine, choline, and oleic acid can be administered to a subject twice a day for at least a week, twice a day for at least two weeks, twice a day for at least a month, twice a day for at least 6 months, or twice a day for a year or more. Within the context of providing a dose to a subject, every day is intended to reflect a subject who has been instructed to be administered the active ingredients every day, and who actually is administered the active ingredients for at least 90% of the days during the desired period of administration.

In some embodiments, the combination of phosphatidyl serine, choline, and oleic acid is chronically administered. “Chronically administering” refers, in one embodiment, to regular administration which is provided indefinitely. In other embodiments, the term refers to regular administration for a significant period of time. For example, in different embodiments chronic administration can include regular administration for at least one month, regular administration for at least 6 weeks, regular administration for at least two months, regular administration for at least 3 months, regular administration for at least 4 months, regular administration for at least 5 months, regular administration for at least 6 months, or regular administration for at least 9 months. In further embodiments, the chronic administration refers to regular administration for at least 1 year, regular administration for at least 1.5 years, regular administration for at least 2 years, or regular administration for more than 2 years. Regular administration refers to administration according to a schedule where it is intended that the subject will receive the active ingredient at regular intervals.

As used herein, “regular intervals” refers to administration in a repeating, periodic fashion where the time between administrations is approximately the same. In various embodiments, administration at regular intervals includes daily administration or weekly administration. In further embodiments, the term refers to administration 1-2 times per week, administration 1-3 times per week, administration 2-3 times per week, administration 1-4 times per week, administration 1-5 times per week, administration 2-5 times per week, administration 3-5 times per week, administration 1-2 times per day, administration 1-3 times per day, administration 1-4 times per day, administration 2-3 times per day, administration 2-4 times per day, administration 3-4 times per day, administration 2-5 times per day, administration 3-5 times per day, or administration 4-5 times per day.

Formulations include those suitable for oral administration. Oral administration, as defined herein, includes any form of administration in which the active ingredients pass through the esophagus of the subject. For example, oral administration includes nasogastric intubation, in which a tube is run from through the nose to the stomach of the subject to administer food or drugs.

Product forms for oral formulations include liquids, powders, solids, semi-solids, semi-liquids compositions, provided that such a formulation allows for the safe and effective oral delivery of the active ingredients and optional nutritive components. In certain embodiments, the oral formulation is a nutritional composition. A nutritional composition is a composition that is edible and includes additional nutrients beyond the active ingredients, such as vitamins, carbohydrates, fats, and proteins. Formulations suitable for oral administration may be presented as discrete units such as tablets, troches, capsules, lozenges, wafers, or cachets, each containing a predetermined amount of phosphatidyl serine, choline, and oleic acid as a powder or granules or as a solution or suspension in an aqueous liquor or non-aqueous liquid such as a syrup, an elixir, an emulsion, or a draught. In certain embodiments, the oral formulation is a nutritional composition that is provided in the form of a liquid, a powder, a solid, a semi-solid or a semi-liquid.

The term “nutritional liquid” as used herein, unless otherwise specified, refers to nutritional compositions in ready-to-drink liquid form, concentrated form, and nutritional liquids made by reconstituting the nutritional powders described herein prior to use. The nutritional liquid may also be formulated as a suspension, an emulsion, a solution, and so forth.

The term “nutritional powder” or “reconstitutable powder” as used herein, unless otherwise specified, refers to nutritional compositions in flowable or scoopable form that can be reconstituted with water or another aqueous liquid prior to consumption and includes both spray dried and drymixed/dryblended powders.

The term “nutritional solid,” as used herein, unless otherwise specified, refers to nutritional compositions that are generally solid in form, and, hence, non-flowable and non-pourable. Some solid examples include bars and sticks.

The term “nutritional semi-solid,” as used herein, unless otherwise specified, refers to nutritional compositions that are intermediate in properties, such as rigidity, between solids and liquids. Some semi-solids examples include puddings, yogurts, gels, gelatins, and doughs.

The term “nutritional semi-liquid,” as used herein, unless otherwise specified, refers to nutritional compositions that are intermediate in properties, such as flow properties, between liquids and solids. Some semi-liquids examples include thick shakes, liquid yogurts, and liquid gels.

The concentration of the combination of phosphatidyl serine, choline, and oleic acid (in total) in the nutritional composition may range up to about 10%, including from about 0.01% to about 10%, and also including from about 0.1% to about 5% and also including from about 0.5% to about 2%, and also including from about 0.4% to about 1.5% by weight of the nutritional liquid composition. In other words, the total amount of PS, C and OA in the liquid nutritional composition includes the foregoing amounts. While the combination of phosphatidyl serine, choline, and oleic acid can be provided in a variety of percent amounts within a nutritional composition, it should be understood that the overall amounts are generally further limited by the gram amounts described below, such that a relatively larger serving size will generally have a lower concentration, in order to avoid providing an excessive amount of the active ingredients. The concentration of the PS, C and OA in a powder nutritional composition will generally be higher in the powder form as compared to the foregoing amounts in the liquid form; however, the concentration of each of PS, C and OA in the powder nutritional composition is generally an amount sufficient to provide the same amount of each in the reconstituted powder when it is ready to be consumed (e.g., up to about 10%, etc., as previously described).

As previously discussed, the nutritional compositions disclosed herein include PS. The PS utilized is preferably in the form of a lecithin phosphatidyl serine complex. Both bovine sources of PS and soy-based sources of PS are available. An example of a suitable commercially available source of PS is Leci-PS™ 30 P available from Cargill, Incorporated or Sharp PS 60FP-IP from Enzymotec, Incorporated. The formulations and nutritional compositions described herein contain PS in an amount sufficient to provide about or exactly 20 mg to 600 mg, 50 mg to 400 mg, 100 mg to 300 mg, or 150 mg to 250 mg of PS per day to the subject being administered the formulation or composition, including, for example, about or exactly 100 mg of PS per day. In certain embodiments, the formulation or nutritional composition is administered to the subject in two servings per day; in certain such embodiments, the formulation or nutritional composition contains PS in an amount of about or exactly 10 mg to 300 mg, 25 to 200 mg, 50 mg to 150 mg, or 75 mg to 125 mg per serving. In other embodiments, the formulation or nutritional composition may be administered to the subject according to a regimen that is other than two times a day, e.g., one time a day, three times a day, four times a day, etc.; it should be understood that the particular amount of PS contained within a serving of the formulation or nutritional composition can be adjusted to account for the administration regimen so that the subject is administered the desired total amount of PS per day, as described above.

The formulations and nutritional compositions described herein also include choline in an amount sufficient to provide about or exactly 20 mg to 600 mg, 50 mg to 550 mg, 100 mg to 450 mg, 125 mg to 200 mg, or 140 mg to 180 mg of choline per day to the subject being administered the formulation or nutritional composition, including, for example, about 150 mg per day or 148 mg of choline per day. In certain embodiments, the formulation or nutritional composition is administered to the subject in two servings per day; in certain such embodiments, the formulation or nutritional composition contains choline in an amount of about or exactly 10 mg to 300 mg, 25 to 275 mg, 50 mg to 225 mg, 62.5 mg to 100 mg, or 70 mg to 90 mg per serving. In other embodiments, the formulation or nutritional composition may be administered to the subject according to a regimen that is other than two times a day, e.g., one time a day, three times a day, four times a day, etc.; it should be understood that the particular amount of choline contained within a serving of the formulation or nutritional composition can be adjusted to account for the administration regimen so that the subject is administered the desired total amount of choline per day, as described above. The choline utilized is generally in the form of a bitartrate, citrate, or chloride salt. Preferably, choline is used in the composition as a choline bitartrate. An example of a suitable commercially available source of choline is available from Balchem Corporation, New Hampton, N.Y.

The formulations and nutritional compositions described herein also include oleic acid in an amount sufficient to provide about or exactly 0.1 g to 30 g, 0.5 g to 25 g, 1.5 g to 15 g, 5 g to 12.5 g, or 7.5 g to 10 g of oleic acid per day to the subject being administered the formulation or nutritional composition, including, for example 9.3 g of oleic acid per day. In certain embodiments, the formulation or nutritional composition is administered to the subject in two servings per day; in certain such embodiments, the formulation or nutritional composition contains oleic acid in an amount of about or exactly 0.1 g to 15 g, 0.25 g to 12.5 g, 0.75 g to 7.5 g, 2.5 g to 6.25 g, or 3.75 g to 5 g per serving. In other embodiments, the formulation or nutritional composition may be administered to the subject according to a regimen that is other than two times a day, e.g., one time a day, three times a day, four times a day, etc.; it should be understood that the particular amount of oleic acid contained within a serving of the formulation or nutritional composition can be adjusted to account for the administration regimen so that the subject is administered the desired total amount of oleic acid per day, as described above. An example of suitable commercial available source of oleic acid is available from Fuji Oil, Singapore.

In certain embodiments, the formulations and nutritional compositions described herein contain PS, C, and OA, in an amount sufficient to provide about or exactly 20 mg to 600 mg of PS, 20 mg to 600 mg of C, and 0.1 g to 30 g of OA per day to the subject being administered the formulation or nutritional composition, including, for example about or exactly 100 mg/day of PS, about 150 mg/day or 148 mg/day of C, and about or exactly 9.3 g/day of OA. In certain other embodiments, the formulations and nutritional compositions described herein contain PS, C, and OA, in an amount sufficient to provide about or exactly 50 mg to 400 mg of PS, 50 mg to 550 mg of C, and 0.5 g to 25 g of OA per day to the subject being administered the formulation or nutritional composition. In yet other embodiments, the formulations and nutritional compositions described herein contain PS, C, and OA, in an amount sufficient to provide about or exactly 100 mg to 300 mg of PS, 100 mg to 450 mg of C, and 1.5 g to 15 g of OA per day to the subject being administered the formulation or nutritional composition. In yet other embodiments, the formulations and nutritional compositions described herein contain PS, C, and OA in an amount sufficient to provide about or exactly 150 mg to 250 mg of PS, 125 mg to 200 mg of C, and 5 g to 12.5 g of OA per day to the subject being administered the formulation or nutritional composition.

In certain embodiments, the formulations and nutritional compositions described herein contain 10 mg to 300 mg of PS, 10 mg to 300 mg of C, and 0.1 to 15 g of OA per serving; in certain such embodiments the formulation or nutritional composition is administered to the subject two times per day. In certain other embodiments, the formulations and nutritional compositions described herein contain 25 mg to 200 mg of PS, 25 mg to 275 mg of C, and 0.25 g to 12.5 g of OA per serving; in certain such embodiments the formulation or nutritional composition is administered to the subject two times per day. In yet other embodiments, the formulations and nutritional composition described herein contain 50 mg to 150 mg of PS, 50 mg to 225 mg of C, and 0.75 g to 7.5 g of OA per serving; in certain such embodiments, the formulation or nutritional composition is administered to the subject two times per day. In yet other embodiments, the formulations and nutritional composition described herein contain 75 mg to 125 mg of PS, 62.5 mg to 100 mg of C, and 2.5 g to 6.25 g of OA per serving; in certain such embodiments, the formulation or nutritional composition is administered to the subject two times per day.

In certain embodiments, the effective amount of each of phosphatidyl serine, choline, and oleic acid is provided to the subject as part of a nutritional composition. The nutritional composition also includes one or more ingredients that help satisfy the subject's nutritional requirements, in addition to providing a useful formulation for the active ingredients. For example, the nutritional composition can include fat, carbohydrate, protein and combinations thereof. In certain embodiments, the nutritional composition includes at least one source of fat, at least one source of carbohydrate, and at least one source of protein. In some embodiments, the nutritional composition can be formulated to provide a specialized nutritional product for use in subjects afflicted with specific diseases or conditions. Many different sources and types of proteins, fats, and carbohydrates are known and can be used in nutritional compositions that include the combination of phosphatidyl serine, choline, and oleic acid. In certain embodiments, the nutritional composition is in the form of a powder suitable for reconstitution to a liquid, a ready-to-drink liquid or a bar.

In certain embodiments, the nutritional composition may be a solid nutritional product. Non-limiting examples of solid nutritional products include snack and meal replacement products, including those formulated as bars; sticks; cookies, breads, cakes or other baked goods; frozen liquids; candy; breakfast cereals; powders, granulated solids or other particulates; snack chips or bites; frozen or retorted entrees; and so forth. In certain embodiments, when the nutritional composition is a solid nutritional product, the serving may be 25 grams to 150 grams.

In certain embodiments, the nutritional composition may be a nutritional liquid. Non-limiting examples of nutritional liquids include snack and meal replacement products, hot or cold beverages, carbonated or non carbonated beverages, juices or other acidified beverages, milk or soy-based beverages, shakes, coffees, teas, enteral feeding compositions, and so forth. Generally, the nutritional liquids are formulated as suspensions or emulsions, but the nutritional liquids can also be formulated in any other suitable forms such as clear liquids, solutions, liquid gels, liquid yogurts, and so forth. In certain embodiments, when the nutritional composition is a liquid nutritional product, the serving may be 150 milliliters to 500 milliliters. In certain other embodiments, when the nutritional composition is a liquid, the serving is 237 milliliters (˜8 fl. oz.). In other embodiments, when the nutritional composition is a liquid, the serving is 177 milliliters to 417 milliliters (˜6 fl. oz. to ˜14 fl. oz.). In yet other embodiments, when the nutritional composition is a liquid, the serving is 207 milliliters to 296 milliliters (˜7 fl. oz. to ˜10 fl. oz.).

In yet other embodiments, the nutritional composition may be formulated as semi-solid or semi-liquid compositions (e.g., puddings, gels, yogurts, etc.), as well as more conventional product forms such as capsules, tablets, caplets, pills, and so forth. In other embodiments, the nutritional composition may be in the form of lozenges, tablets (e.g., chewable, coated, etc.), pastes, gels, or yogurts.

Examples of nutritional composition forms suitable for use herein include snack and meal replacement products, including those formulated as bars, sticks, cookies or breads or cakes or other baked goods, frozen liquids, candy, breakfast cereals, powders or granulated solids or other particulates, snack chips or bites, frozen or retorted entrees, and so forth. The nutritional composition can also be in forms that fall between solid and liquid, such as semi-solid or semi-liquid compositions such as puddings or gels.

Examples of liquid product forms suitable for use herein include snack and meal replacement products, hot or cold beverages, carbonated or non-carbonated beverages, juices or other acidified beverages, milk or soy-based beverages, shakes, coffees, teas, compositions for administration by nasogastric intubation, and so forth. These liquid compositions are most typical formulated as suspensions or emulsions, but can also be formulated in any other suitable form such as clear liquids, substantially clear liquids, liquid gels, and so forth.

The combination of phosphatidyl serine, choline, and oleic acid can be used for the preparation of a medicament for the treatment of cognitive disorder, or for treating a subject in need of cognitive improvement. Use for preparation of a medicament can include any of the various embodiments of the method and composition described herein. For example, the medicament can be prepared for the treatment of dementia or cognitive dysfunction, of for use in a subject has been diagnosed with dementia or cognitive dysfunction. In a further exemplary embodiment, the amounts are as follows: phosphatidyl serine in an amount of from 10 mg to 300 mg, choline in an amount of from 10 mg to 300 mg, and oleic acid in an amount of from 0.1 g to 15 g in the medicament; phosphatidyl serine oleic acid in an amount of from 25 mg to 200 mg, choline oleic acid in an amount of from 25 mg to 275 mg, and oleic acid oleic acid in an amount of from 0.25 g to 12.5 g in the medicament; phosphatidyl serine oleic acid in an amount of from 50 mg to 150 mg, choline oleic acid in an amount of from 50 mg to 225 mg, and oleic acid oleic acid in an amount of from 0.75 g to 7.5 g in the medicament; or phosphatidyl serine oleic acid in an amount of from 75 mg to 125 mg, choline oleic acid in an amount of from 62.5 mg to 100 mg, and oleic acid oleic acid in an amount of from 2.5 g to 6.25 g in the medicament.

The nutritional composition includes one or more ingredients that help satisfy the subjects' nutritional requirements. The optional nutrients can provide up to about 1000 kcal of energy per serving or dose, including from about 25 to about 900 kcal, from about 75 to about 700 kcal, from about 150 to about 500 kcal, from about 150 to about 350 kcal, or from about 200 to about 350 kcal per serving.

In certain embodiments, the nutritional composition may comprise 8 grams to 100 grams of protein per serving or 10 grams to 100 grams of protein per serving. In other embodiments, the nutritional composition may comprise 8 grams to 50 grams of protein per serving. In still other embodiments, the nutritional composition may comprise 8 grams to 25 grams of protein per serving. Virtually any source of protein may be used so long as it is suitable for use in oral nutritional compositions and is otherwise compatible with any other selected ingredients or features in the nutritional composition.

The source of protein may include, but is not limited to, intact, hydrolyzed, and partially hydrolyzed protein, which may be derived from any known or otherwise suitable source such as milk (e.g., casein, whey), animal (e.g., meat, fish), cereal (e.g., rice, corn), vegetable (e.g., soy, pea), and combinations thereof. Non-limiting examples of the source of protein include milk protein isolates, milk protein concentrates, casein protein isolates, whey protein concentrates, whey protein isolates, whey protein hydrolysates, sodium or calcium caseinates, whole cow's milk, partially or completely defatted milk, soy protein isolates, soy protein concentrates, soy protein hydrolysates, pea protein concentrates, pea protein isolates, pea protein hydrolysates, and so forth. In addition, the nutritional composition may include from 8 grams to 100 grams of protein per serving, and can comprise any one source of protein or any combination of any of the various sources of protein listed above.

In certain embodiments, the nutritional composition may include at least one source of fat. In other embodiments, the nutritional composition may include no fat, or essentially no fat (i.e., less than 0.5 grams of fat per serving). In some embodiments where the nutritional composition contains fat, the nutritional composition may comprise 2 grams to 45 grams of at least one source of fat per serving. In other embodiments, the nutritional composition may comprise 5 grams to 35 grams of at least one source of fat per serving. In yet other embodiments, the nutritional composition may comprise 15 grams to 30 grams of at least one source of fat per serving.

In general, any source of fat may be used so long as it is suitable for use in oral nutritional compositions and is otherwise compatible with any other selected ingredients or features present in the nutritional composition. The source of fat may be derived from plants, animals, and combinations thereof. Non-limiting examples of suitable sources of fat for use in the nutritional compositions described herein include coconut oil, fractionated coconut oil, soy oil, corn oil, olive oil, safflower oil, high oleic safflower oil, MCT (medium chain triglycerides) oil, sunflower oil, high oleic sunflower oil, palm oil, palm kernel oil, palm olein, canola oil, marine oils, cottonseed oils, and combinations thereof.

In certain embodiments, the nutritional composition may include at least one source of carbohydrate. In some embodiments, the nutritional composition may comprise 15 grams to 110 grams of at least one source of carbohydrate per serving. In other embodiments, the nutritional composition may comprise 25 grams to 90 grams of at least one source of carbohydrate per serving. In yet other embodiments, the nutritional composition may comprise 40 grams to 65 grams of at least one source of carbohydrate per serving.

The at least one source of carbohydrate suitable for use in the nutritional compositions disclosed herein may be simple, complex, or variations or combinations thereof. Generally, any source of carbohydrate may be used so long as it is suitable for use in oral nutritional compositions and is otherwise compatible with any other selected ingredients or features present in the nutritional composition. Non-limiting examples of a source of carbohydrate suitable for use in the nutritional emulsions described herein may include maltodextrin, hydrolyzed or modified starch or cornstarch, glucose polymers, corn syrup, corn syrup solids, rice-derived carbohydrates, sucrose, glucose, fructose, lactose, high fructose corn syrup, honey, sugar alcohols (e.g., maltitol, erythritol, sorbitol), and combinations thereof.

The amount or concentration of the at least one source of fat, at least one source of protein, and at least one source of carbohydrate present in certain embodiments of the nutritional compositions described herein may vary widely depending on the product formulation of the nutritional composition (e.g., solid product, liquid). In addition, the amount or concentration of the at least one source of fat, at least one source of protein, and at least one source of carbohydrate present in certain embodiments of the nutritional compositions described herein may be characterized based upon: (i) a percentage of the total calories per serving in the nutritional composition; or (ii) the total weight of each ingredient present in a serving of the nutritional composition; or both (i) and (ii). For example, in certain embodiments, the amount or concentration of the at least one source of fat, at least one source of protein, and the at least one source of carbohydrate present in the nutritional composition can be within the ranges shown in the examples provided in Tables I and II below. As previously discussed, in other embodiments, the nutritional composition contains no fat or essentially no fat (i.e., less than 0.5 grams per serving).

TABLE I

| Nutrient (% total | | | |
|---|---|---|---|
| calories) | Example A | Example B | Example C |
| Carbohydrate | 0-94 | 10-85 | 20-60 |
| Fat | 0-94 | 5-80 | 20-60 |
| Protein | 6-100 | 10-85 | 20-60 |

TABLE II

| Nutrient (grams per | | | |
|---|---|---|---|
| serving) | Example D | Example E | Example F |
| Carbohydrate | 15-110 | 25-90 | 40-65 |
| Fat | 2-45 | 5-35 | 15-30 |
| Protein | 8-100 | 8-50 | 8-30 |

In certain embodiments, the nutritional composition comprises at least one source of fat and at least one source of carbohydrate, and the at least one source of fat provides 5 percent to 80 percent of the caloric density per serving and the at least one source of carbohydrate provides 10 percent to 85 percent of the caloric density per serving. In other embodiments, the nutritional composition comprises at least one source of fat and at least one source of carbohydrate, and the at least one source of fat provides 10 percent to 65 percent of the caloric density per serving and the at least one source of carbohydrate provides 20 percent to 75 percent of the caloric density per serving. In yet other embodiments, the nutritional composition comprises at least one source of fat and at least one source of carbohydrate, and the at least one source of fat provides 30 percent to 50 percent of the caloric density per serving and the at least one source of carbohydrate provides 30 percent to 50 percent of the caloric density per serving. Such embodiments provide flexibility in formulating calorie dense nutritional compositions with various other ingredients.

The nutritional composition can also include other ingredients that may modify the physical, nutritional, chemical, hedonic, or processing characteristics of the product or serve as pharmaceutical or additional nutritional components. Non-limiting examples of such optional ingredients include preservatives, antioxidants, emulsifying agents, buffers, fructooligosaccharides, chromium picolinate, pharmaceutical additives, colorants, flavors or masking agents, thickening agents and stabilizers, artificial sweeteners, hydrocolloids such as guar gum, xanthan gum, carrageenan, gellan gum, gum acacia, and so forth.

The nutritional composition can also include vitamins, minerals, and combinations thereof. Exemplary vitamins include, but are not limited to, vitamin A, vitamin E, vitamin D2, vitamin D3, vitamin A palmitate, vitamin E acetate, vitamin C palmitate (ascorbyl palmitate), vitamin K, thiamine, riboflavin, pyridoxine, vitamin B12, carotenoids (e.g., beta-carotene, zeaxanthin, lutein, lycopene), niacin, folic acid, pantothenic acid, biotin, vitamin C, choline, inositol, salts and derivatives thereof, and combinations thereof. Exemplary minerals include, but are not limited to, calcium, selenium, potassium, iodine, phosphorus, magnesium, iron, zinc, manganese, copper, sodium, molybdenum, chromium, chloride, and combinations thereof.

The various embodiments of the nutritional composition may be prepared by any process or suitable method (now known or known in the future) for making a selected product form, such as a nutritional solid, a nutritional powder, or a nutritional liquid. Many such techniques are known for any given product form such as nutritional liquids or nutritional powders and can easily be applied by one of ordinary skill in the art to the various embodiments of the nutritional composition according to the first, second, third, and fourth embodiments disclosed herein.

Liquid nutritional compositions can be manufactured by any process or suitable method for making nutritional emulsions. In one suitable manufacturing process, at least three separate slurries are prepared. These slurries include: a protein-in-fat (PIF) slurry, a carbohydrate-mineral (CHO-MIN) slurry and a protein-in-water (PIW) slurry. The PIF slurry is formed by heating and mixing any oils that are selected for the fat component (when present) and then adding an emulsifier (e.g., lecithin), fat-soluble vitamins and a portion of the total protein (preferably about half of the milk protein concentrate) with continued heat and agitation. The CHO-MN slurry is formed by adding to water (with heat and agitation), minerals (e.g., potassium citrate, dipotassium phosphate, sodium citrate, etc.), trace and ultra trace minerals (often as a pre-mix), and thickening-type or suspending agents (e.g., Avicel, gellan, carragenan). The CHO-MIN slurry that results is held for 10 minutes with continued heat and agitation and then additional minerals may be added (e.g., potassium chloride, magnesium carbonate, potassium iodide, etc.) and/or carbohydrates (e.g., fructooligosaccharides, sucrose, corn syrup, etc.). The PIW slurry is formed by mixing the remaining protein (i.e., sodium caseinate, soy protein, why protein, etc.) into water.

The three slurries are blended together with heat and agitation and the pH is adjusted to the desired range (typically near neutral, around 6.6-7), after which the composition is subjected to high-temperature short-time (HTST) processing during which time the composition is heat treated, emulsified and homogenized and allowed to cool. Water soluble vitamins and ascorbic acid are added (if applicable), the pH is again adjusted (if necessary), flavors are added and any additional water can be added to adjust the solids content to the desired range.

A nutritional powder, such as a spray dried nutritional powder or drymixed nutritional powder, may be prepared by any collection of known or otherwise effective technique, suitable for making and formulating a nutritional powder.

For example, when the nutritional powder is a spray dried nutritional powder, the spray drying step may likewise include any spray drying technique that is known for or otherwise suitable for use in the production of nutritional powders. Many different spray drying methods and techniques are known for use in the nutrition field, all of which are suitable for use in the manufacture of the spray dried nutritional powders herein.

One method of preparing the spray dried nutritional powder comprises forming and homogenizing an aqueous slurry or liquid comprising predigested fat, and optionally protein, carbohydrate, and other sources of fat, and then spray drying the slurry or liquid to produce a spray dried nutritional powder. The method may further comprise the step of spray drying, drymixing, or otherwise adding additional nutritional ingredients, including any one or more of the ingredients described herein, to the spray dried nutritional powder.

Other suitable methods for making nutritional products are described, for example, in U.S. Pat. No. 6,365,218 (Borschel, et al.), U.S. Pat. No. 6,589,576 (Borschel, et al.), U.S. Pat. No. 6,306,908 (Carlson, et al.), U.S. Pat. Appl. No. 20030118703 A1 (Nguyen, et al.), which descriptions are incorporated herein by reference to the extent that they are consistent herewith.

The following examples are included for purposes of illustration and are not intended to limit the scope of the invention.

### EXAMPLES

### Example 1

### Liquid Formulation

An exemplary nutritional composition suitable for formulating a combination of phosphatidyl serine, choline, and oleic acid is described in Table III below, with the specific ingredients provided immediately thereafter.

TABLE III
Liquid Formulation Information

| | UNIT | per 8 fl oz |
|---|---|---|
| Energy EU | Kcal | 350 |
| Protein | G | 20 |
| Fat | G | 11 |
| Linoleic acid | G | 3 |
| Carbohydrate | G | 44 |
| Fructooligosaccharide | G | 3 |
| Sugar | G | 20 |
| Phosphatidyl Serine | Mg | 50 |
| Choline | Mg | 74 |
| Oleic acid | G | 4.65 |
| VITAMINS | | |
| Vitamin A (Palmitate) | IU | 1000 |
| Vitamin D₃ | IU | 160 |
| Vitamin E | IU | 30 |
| Vitamin K₁ | Mcg | 20 |
| Vitamin C | Mg | 60 |
| Folic Acid | Mcg | 200 |
| Vitamin B₁ | Mg | 0.38 |
| Vitamin B₂ | Mg | 0.43 |
| Vitamin B₆ | Mg | 0.5 |
| Vitamin B₁₂ | Mcg | 3 |
| Niacin | Mg | 5 |
| Pantothenate | Mg | 2.5 |
| Biotin | Mcg | 75 |
| L-carnitine | Mg | 43 |
| MINERALS | | |
| Sodium | Mg | 240 |
| Potassium | Mg | 560 |
| Chloride | Mg | 150 |
| Calcium | Mg | 500 |
| Phosphorus | Mg | 350 |
| Magnesium | Mg | 100 |
| Iron | Mg | 4.5 |
| Zinc | Mg | 15 |
| Manganese | Mg | 0.50 |
| Copper | Mg | 0.50 |
| Iodine | Mcg | 25 |
| Selenium | Mcg | 30 |
| Chromium | Mcg | 30 |
| Molybdenum | Mcg | 30 |

Note:
In Tables III, IV, and V, Mg = milligrams, G = grams, Mcg = micrograms, Kcal = kilocalories and IU = international units.

The nutritional composition described in Table III includes Water, Corn syrup, Sucrose, Milk Protein Concentrate, Sodium Caseinate, Canola Oil, Corn Oil, Oleic Acid, Fructooligosaccharides, Soy Protein Isolate, Whey Protein Concentrate, Potassium Citrate, Natural and Artificial Flavors, Potassium Phosphate, Lecithin, Cellulose Gel, Magnesium Hydroxide, Calcium Carbonate, Ascorbic Acid, Calcium Phosphate, Choline Chloride, Phosphatidyl Serine, Sodium Chloride, Sodium Phosphate, Potassium Hydroxide, Zinc Sulfate, Cellulose Gum, L-Carnitine, Carrageenan, dl-Alpha-Tocopheryl Acetate, Dextrose, Ferrous Sulfate, Maltodextrin, Niacinamide, Gellan Gum, Calcium Pantothenate, Citric Acid, Cupric Sulfate, Manganese Sulfate, Chromium Chloride, Thiamine Chloride Hydrochloride, Coconut Oil, Vitamin A Palmitate, Pyridoxine Hydrochloride, Riboflavin, Folic Acid, Biotin, Sodium Selenate, Sodium Molybdate, Potassium Iodide, Phylloquinone, Cyanocobalamin, and Vitamin D3.

### Example 2

### Diabetic Liquid Formulation

An exemplary liquid nutritional composition formulated for use with diabetic subjects and suitable for formulating a combination of phosphatidyl serine, choline, and oleic acid is provided in Table IV below, with the specific ingredients provided immediately thereafter.

TABLE IV
Diabetic Formulation Information

| | UNIT | per 8 fl oz |
|---|---|---|
| Nutrient Density | Cal/ml | 0.93 |
| Protein | % Cal | 18 |
| Carbohydrate | % Cal | 47 |
| Fat | % Cal | 35 |
| Osmolality | mOsm/L | 86 |
| Viscosity | | Thin |
| Protein | G | 9.9 |
| Carbohydrate | G | 29.3 |
| Fat | G | 8.6 |
| Water | G | 200 |
| Phosphatidyl Serine | Mg | 50 |
| Choline | Mg | 74 |
| Oleic acid | G | 4.65 |
| VITAMINS | | |
| Vitamin A (Palmitate) | IU | 1750 |
| Vitamin A (B- | Mg | 0.5 |
| Carotene) | | |
| Vitamin D | IU | 100 |
| Vitamin E | IU | 30 |
| Vitamin K | Mcg | 20 |
| Vitamin C | Mg | 60 |
| Folic Acid | Mcg | 200 |
| Vitamin B₁ | Mg | 0.38 |
| Vitamin B₂ | Mg | 0.43 |
| Vitamin B₆ | Mg | 1.0 |
| Vitamin B₁₂ | Mcg | 3.0 |
| Niacin | Mg | 5.0 |
| Pantothenate | Mg | 2.5 |
| Biotin | Mcg | 75 |
| L-carnitine | Mg | 43 |
| MINERALS | | |
| Sodium | Mg | 210 |
| Potassium | Mg | 370 |
| Chloride | Mg | 355 |
| Calcium | Mg | 250 |
| Phosphorus | Mg | 250 |
| Magnesium | Mg | 100 |
| Iron | Mg | 4.5 |
| Zinc | Mg | 3.8 |
| Manganese | Mg | 1.0 |
| Copper | Mg | 0.50 |
| Iodine | Mcg | 38 |
| Selenium | Mcg | 18 |
| Chromium | Mcg | 120 |
| Molybdenum | Mcg | 38 |

The liquid nutritional composition described in Table IV includes Water, Corn Maltodextrin, Sodium & Calcium Caseinates, Maltitol Syrup, High Oleic Safflower Oil, Fructose, Soy Protein Isolate, Soy Fiber, Short-Chain Fructooligosaccharides, Canola Oil, Oleic Acid, Calcium Phosphate, Magnesium Chloride, Soy Lecithin, Artificial Flavor, Sodium Citrate, Magnesium Phosphate, Potassium Citrate, Potassium Chloride, Potassium Phosphate, Ascorbic Acid, Choline Chloride, Phosphatidyl Serine, dl-Alpha-Tocopheryl Acetate, Gellan Gum, Acesulfame Potassium, Ferrous Sulfate, Zinc Sulfate, Niacinamide, Manganese Sulfate, Calcium Pantothenate, Cupric Sulfate, Sucralose, Pyridoxine Hydrochloride, Thiamine Chloride Hydrochloride, Vitamin A Palmitate, Riboflavin, Chromium Chloride, Beta-Carotene, Folic Acid, Biotin, Sodium Molybdate, Potassium Iodide, Sodium Selenate, Phylloquinone, Cyanocobalamin, and Vitamin D3.

### Example 3

### Fat-free Liquid Formulation

An exemplary liquid nutritional composition containing essentially no fat, having a clear, juice-like appearance and an acidic pH suitable for formulating a combination of phosphatidyl serine, choline, and oleic acid is provided in Table V below, with the specific ingredients provided immediately thereafter.

TABLE V
Clear Formulation Information

| | UNIT | per 10 fl oz |
|---|---|---|
| Nutrient Density | Cal/ml | 0.61 |
| Protein | % Cal | 20 |
| Carbohydrate | % Cal | 80 |
| Fat | % Cal | 0 |
| Osmolality | mOsm/L | |
| Viscosity | | Thin |
| Protein | G | 9 |
| Carbohydrate | G | 35 |
| Fat | G | 0 |
| Phosphatidyl Serine | Mg | 50 |
| Choline | Mg | 74 |
| Oleic acid | G | 4.65 |
| VITAMINS | | |
| Vitamin A | % DV | 30 |
| Vitamin D | % DV | 15 |
| Vitamin E | % DV | 30 |
| Vitamin K | % DV | 30 |
| Vitamin C | % DV | 45 |
| Thiamin | % DV | 30 |
| Folic Acid | % DV | 15 |
| Riboflavin | % DV | 20 |
| Vitamin B₆ | % DV | 20 |
| Vitamin B₁₂ | % DV | 20 |
| Niacin | % DV | 10 |
| Pantothenic acid | % DV | 8 |
| Biotin | % DV | 10 |
| MINERALS | | |
| Calcium | % DV | 6 |
| Phosphorus | % DV | 20 |
| Magnesium | % DV | 2 |
| Iron | % DV | 15 |
| Zinc | % DV | 30 |
| Manganese | % DV | 50 |
| Copper | % DV | 15 |
| Iodine | % DV | 35 |
| Selenium | % DV | 20 |
| Chromium | % DV | 15 |
| Molybdenum | % DV | 50 |

The liquid nutritional composition described in Table V includes Water, Corn Syrup Solids, Sugar, Whey Protein Isolate and less than 0.5% of the following: Oleic Acid, Phosphatidyl Serine, Choline Chloride, Citric Acid, Natural & Artificial Flavor, Phosphoric Acid, Ascorbic Acid, Acesulfame Potassium, Sucralose, Zinc Sulfate, dl-Alpha-Tocopheryl Acetate, Ferrous Sulfate, Niacinamide, Manganese Sulfate, Calcium Pantothenate, Cupric Sulfate, FD&C Yellow #6, Vitamin A Palmitate, Thiamine Chloride Hydrochloride, Pyridoxine Hydrochloride, FD&C Red #40, Riboflavin, Folic Acid, Chromium Chloride, Sodium Molybdate, Biotin, Potassium Iodide, Sodium Selenate, Phylloquinone, Vitamin D3, and Cyanocobalamin.

### Example 4

In this Example, the administration of choline chloride (alone) on NMDAR dependent hippocampal LTP in mice was analyzed. As used herein the term AMPA is an acronym for α-amino-3-hydroxy-5-methyl-4-isoxazolepropionic acid; the terms AMPA receptor and AMPAR are used interchangeably. As used herein the term NDMA is an acronym for N-Methyl-D-aspartic acid or N-Methyl-D-aspartate; the terms NDMA receptor and NDMAR are used interchangeably.

Long lasting changes in the strength of AMPA and NMDA receptor mediated glutamatergic synaptic transmission in the hippocampus, known as hippocampal synaptic plasticity, is a key underlying molecular mechanism in learning and memory. AMPA and NMDA dependant hippocampal synaptic plasticity is associated with trafficking and delivery of AMPA receptors at the synapse in response to NMDAR activation. Previous studies have shown that in cognitive decline associated with aging or Alzheimer's disease onset, the strength of AMPA and NMDA synaptic transmission is compromised due to the internalization of NMDA and AMPA receptors, leading to deficits in NMDAR and AMPAR expression at the synapse. During learning, neuronal activation leads to pre-synaptic release of glutamate resulting in the activation of post-synaptic AMPA and NMDA receptors. Activation of post-synaptic AMPA receptors results in the flow of Na⁺ ions, causing the depolarization of post-synaptic neuron. This depolarization abolishes the blocking of NMDA receptors by Mg²⁺, resulting in the opening of NMDA receptor channels for Na⁺ and Ca²⁺ ions. Ca²⁺ influx through NMDA receptors is known to trigger intracellular signaling cascades, notably the phosphorylation of Ca²⁺/calmodulin-dependent protein kinase (CaMKII) that phosphorylates AMPA receptors and modulates the channel properties, ultimately resulting in long-term potentiation (LTP), the physiological correlate of synaptic plasticity (learning and memory).

Therefore in this Example, NMDAR dependant hippocampal LTP was used as a physiological marker to assess the efficacy of choline chloride in improving or enhancing cognitive function. To evaluate the efficacy of choline in improving or enhancing cognitive function, NMDAR dependant LTP was recorded on hippocampal brain slices in vitro.

Experiments were carried out with 7-9 weeks-old C57/Black6 mice (from Elevage Janvier, Le Genest St Isle, France). Animals were housed and used in accordance with the French and European legislations for animal care. The mice were sacrificed by fast decapitation, without previous anaesthesia. The brain was quickly removed and soaked in ice-cold oxygenated buffer having the following composition:

| Cutting Solution | Final concentration (mM) |
|---|---|
| KCl | 2 |
| NaH₂PO₄ | 1.2 |
| MgCl₂ | 7 |
| CaCl₂ | 0.5 |
| NaHCO₃ | 26 |
| D-glucose | 11 |
| Saccharose | 250 |

Hippocampus slices (350 micrometer) were cut with a MacIlwain tissue-chopper and incubated at room temperature for at least 60 minutes in Artificial Cerebro-Spinal Fluid (ACSF) having the following composition:

| ACSF | Final concentration (mM) |
|---|---|
| NaCl | 126 |
| KCl | 3.5 |
| NaH₂PO₄ | 1.2 |
| MgCl₂ | 1.3 |
| CaCl₂ | 2 |
| NaHCO₃ | 25 |
| D-glucose | 11 |

During experiments, slices were continuously perfused with oxygenated ACSF.

Choline chloride (powder form; Molecular weight=139.62 grams/mole) was dissolved as a 100 millimolar stock solution in Milli-Q water, aliquoted and stored at −20° C. until use. Aliquots were thawed and vortexed each day of experiment and then diluted into ACSF to reach the final concentration. D(−)-2-Amino-5-phosphonovaleric acid (D-AP5) (ref. Ab120003, Abcam, batch: APN11040-2-2) was dissolved as a 30 millimolar stock solution in Milli-Q water, aliquoted and stored at −20° C. until use. Aliquots were thawed and vortexed each day of experiment and then 1000× diluted into ACSF to reach the final concentration of 30 micromolar. 2,3-dihydroxy-6-nitro-7-sulfamoyl-benzo [f]quinoxaline-2,3-dione (NBQX) (ref: Ab120045, Abcam, batch: APN10009-4-1) was dissolved as a 10 millimolar stock solution in DMSO (dimethylsulfoxide), aliquoted and stored at −20° C. until use. Aliquots were thawed and vortexed each day of experiment and then 1000× diluted into ACSF to reach the final concentration of 10 micromolar.

All data was recorded with a MEA set-up, commercially available from MultiChannel Systems MCS GmbH (Reutlingen, Germany), and composed of a 4-channel stimulus generator and a 60-channel amplifier head-stage connected to a 60-channels A/D card. Software for stimulation, recordings and analysis were commercially available from Multi Channel Systems: MC Stim (3.2.4 release) and MC Rack (4.0.0 release), respectively. All of the experiments were carried out with 3-dimensional MEA (Ayanda Biosystems, S.A., CH-1015 Lausanne, Switzerland) consisting of 60 tip-shaped and 60-μm-high electrodes spaced by 100 μm. The MEA electrodes were made of platinum with kΩ450<impedance<600 kΩ.

A 350-μm thick mouse hippocampal slice was disposed on the multi-electrode array (100 μm distant electrodes). One electrode was chosen to stimulate Schaeffer collaterals at the CA3/CA1 interface. An I/O curve was performed to monitor evoked-responses for stimulations between 100 and 800 μA (micro-Amperes), by 100 μA steps. The stimulus was a monopolar biphasic current pulse (negative for 60 microseconds and then positive for 60 microseconds), set to evoke 40% of the maximal amplitude response (as determined with the I/O curve) and was applied every 30 seconds to evoke “responses” (i.e., field Excitatory Post Synaptic Potentials; fEPSP) in the CA1 region.

Short term memory formation, accompanied by a weak potentiation, effect of Choline chloride on NMDA dependant LTP induced by a weak tetanus was also analyzed. Following a 10-minute period to verify the baseline stability of fEPSP to elicit a weak potentiation, a weak tetanus (10 stimulations at 100 Hz for 0.1 s at 20% of IMAX (Kanno et al., Brain research, 2004)) was used. Weak tetanus-induced potentiation was followed over an approximately 40-minute period.

Evoked-responses (fEPSP) were recorded if they satisfied quality criteria described in Standard Operating Procedures: correct location, stable baseline (fluctuation within +/−10% during ten consecutive minutes), amplitude>100 μV after background noise subtraction. The fEPSP from selected electrodes were simultaneously sampled at 5 kHz and recorded on the hard disk of a PC until offline analysis. In parallel, fEPSP amplitudes of selected electrodes were compiled online (with MC Rack program) to monitor and to follow the performance of the experiments. Data was plotted in a standard spreadsheet file for off-line analysis. Since fEPSPs result from glutamatergic synaptic transmission consecutive to afferent pathway stimulation, at the end of each experiment, 10 μM NBQX were perfused on the slice to validate the glutamatergic nature of synaptic transmission as well as to subtract background noise at individual electrode level. Control LTP were recorded in parallel, with hippocampal slices prepared from the same animals as the ones used to evaluate the compounds.

During the experiments, the slices were continuously perfused with ACSF solutions (bubbled with 95% O₂-5% CO₂) at the rate of 3 milliliters/minute with a peristaltic pump (MEA chamber volume: ˜1 milliliter). Complete solution exchange in the MEA chamber was achieved 20 seconds after the switch of solutions. The perfusion liquid was continuously pre-heated at 37° C. just before reaching the MEA chamber with a heated-perfusion cannula (PH01, MultiChannel Systems, Reutlingen, Germany). The temperature of the MEA chamber was maintained at 37° C.+/−0.1° C. with a Peltier element located in the MEA amplifier headstage.

I/O curves from identical experimental conditions were analysed using repeated measure two-way ANOVA followed by a post hoc Bonferroni test. LTP from identical experimental conditions were analysed using one-way ANOVA followed by a post hoc Dunnett test. Statistical analysis was performed by using Prism 5.0 software. A critical P value of P<0.05 was considered significant for the statistical tests used throughout the study.

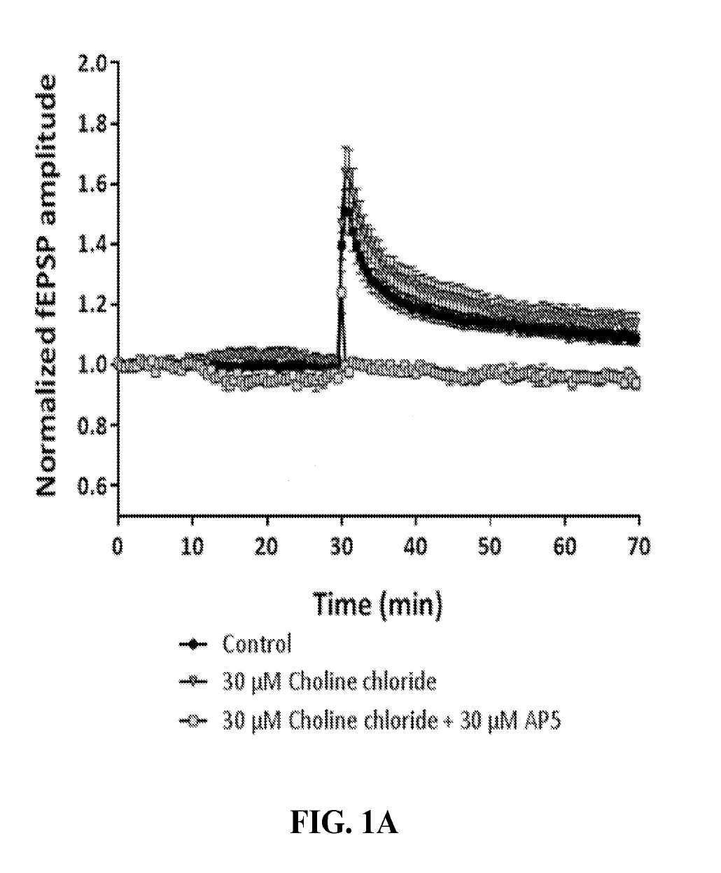

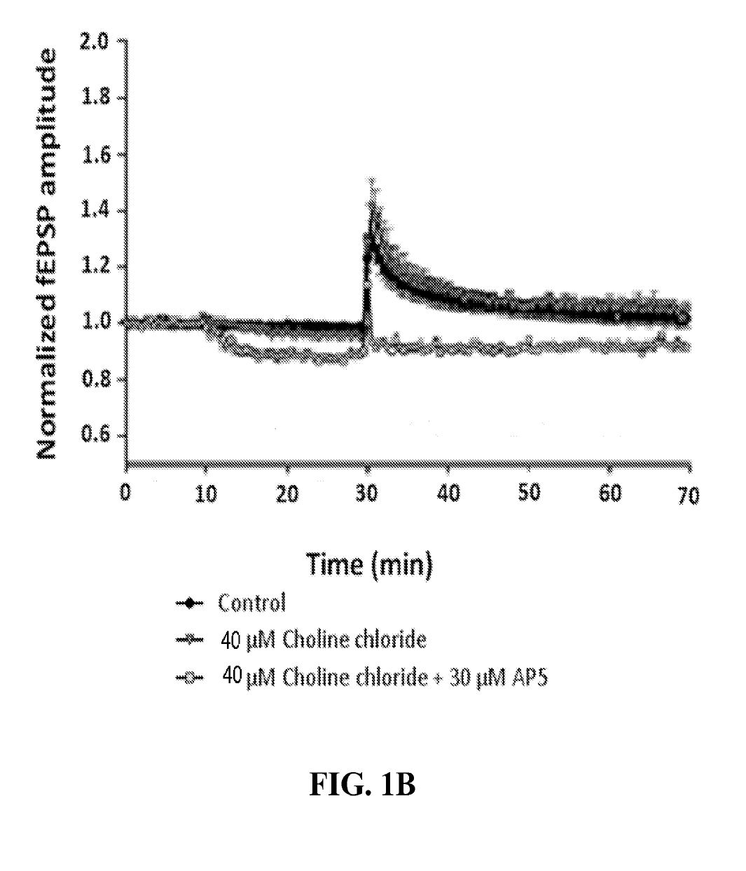

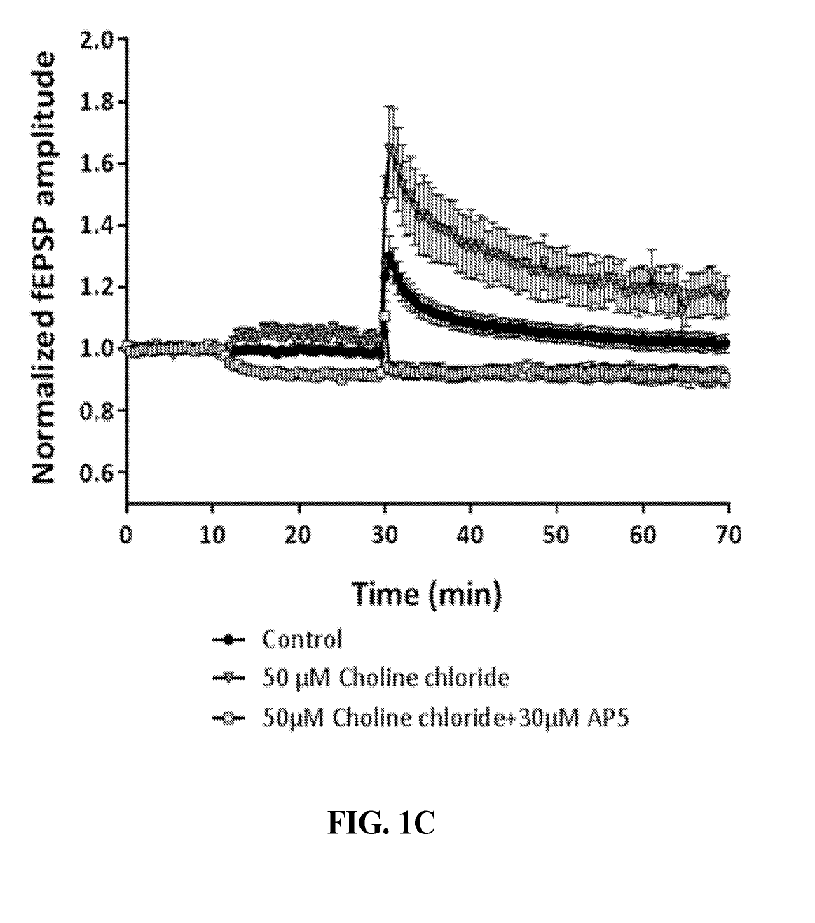

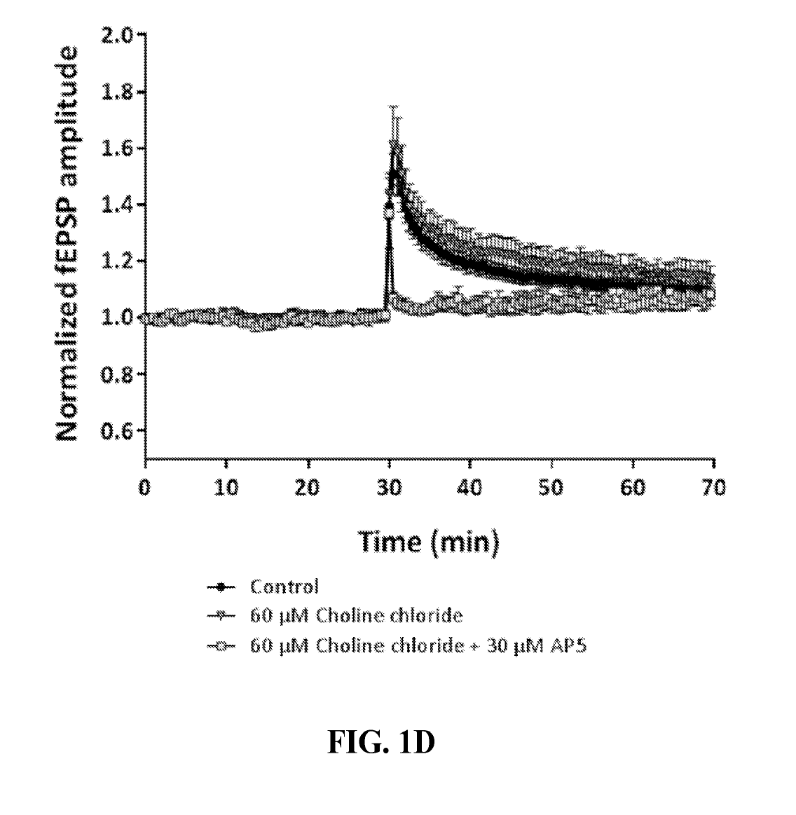

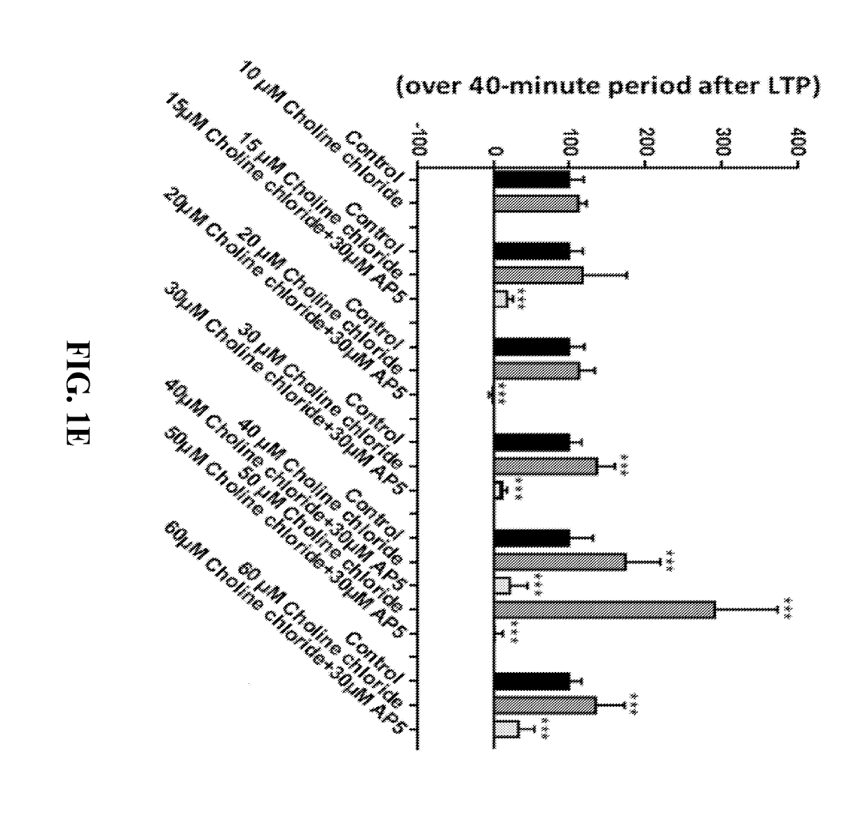

As shown in FIGS. 1A-1E, choline chloride at 30, 40 and 50 micromolar significantly enhanced NMDAR dependant hippocampal LTP induced by weak tetanic stimulation (10 stimulations applied at 100 Hz for 0.1 seconds). At 10, 15 and 20 micromolar Choline Chloride did not produce any effect on NMDAR dependant hippocampal LTP. Weak stimulation resulted in weak potentiation such as that which normally occurs during short term memory formation.

### Example 5

In this Example, the administration of oleic acid (Ethyl ester; Molecular weight=310.51 g/mol) on NMDAR dependent hippocampal LTP in mice was analyzed. As used herein the term AMPA is an acronym for α-amino-3-hydroxy-5-methyl-4-isoxazolepropionic acid; the terms AMPA receptor and AMPAR are used interchangeably. As used herein the term NDMA is an acronym for N-Methyl-D-aspartic acid or N-Methyl-D-aspartate; the terms NDMA receptor and NDMAR are used interchangeably.

Long lasting changes in the strength of AMPA and NMDA receptor mediated glutamatergic synaptic transmission in the hippocampus, known as hippocampal synaptic plasticity, is a key underlying molecular mechanism in learning and memory. AMPA and NMDA dependant hippocampal synaptic plasticity is associated with trafficking and delivery of AMPA receptors at the synapse in response to NMDAR activation. Previous studies have shown that in cognitive decline associated with aging or Alzheimer's disease onset, the strength of AMPA and NMDA synaptic transmission is compromised due to the internalization of NMDA and AMPA receptors, leading to deficits in NMDAR and AMPAR expression at the synapse. During learning, neuronal activation leads to pre-synaptic release of glutamate resulting in the activation of post-synaptic AMPA and NMDA receptors. Activation of post-synaptic AMPA receptors results in the flow of Na⁺ ions, causing the depolarization of post-synaptic neuron. This depolarization abolishes the blocking of NMDA receptors by Mg²⁺, resulting in the opening of NMDA receptor channels for Na⁺ and Ca²⁺ ions. Ca²⁺ influx through NMDA receptors is known to trigger intracellular signaling cascades, notably the phosphorylation of Ca²⁺/calmodulin-dependent protein kinase (CaMKII) that phosphorylates AMPA receptors and modulates the channel properties, ultimately resulting in long-term potentiation (LTP), the physiological correlate of synaptic plasticity (learning and memory).

Therefore in this Example, NMDAR dependant hippocampal LTP was used as a physiological marker to assess the efficacy of oleic acid in improving or enhancing cognitive function. To evaluate the efficacy of oleic acid in improving or enhancing cognitive function, NMDAR dependant LTP was recorded on hippocampal brain slices in vitro.

Experiments were carried out with 7-9 weeks-old C57/Black6 mice (from Elevage Janvier, Le Genest St Isle, France). Animals were housed and used in accordance with the French and European legislations for animal care. The mice were sacrificed by fast decapitation, without previous anaesthesia. The brain was quickly removed and soaked in ice-cold oxygenated buffer having the following composition:

| Cutting Solution | Final concentration (mM) |
|---|---|
| KCl | 2 |
| NaH₂PO₄ | 1.2 |
| MgCl₂ | 7 |
| CaCl₂ | 0.5 |
| NaHCO₃ | 26 |
| D-glucose | 11 |
| Saccharose | 250 |

Hippocampus slices (350 micrometer) were cut with a MacIlwain tissue-chopper and incubated at room temperature for at least 60 minutes in Artificial Cerebro-Spinal Fluid (ACSF) having the following composition:

| ACSF | Final concentration (mM) |
|---|---|
| NaCl | 126 |
| KCl | 3.5 |
| NaH₂PO₄ | 1.2 |
| MgCl₂ | 1.3 |
| CaCl₂ | 2 |
| NaHCO₃ | 25 |
| D-glucose | 11 |

During experiments, slices were continuously perfused with oxygenated ACSF.

Oleic acid ethyl ester (powder form; Molecular weight=310.51 grams/mole) was freshly prepared as a 100 millimolar stock solution in Dimethyl sulfoxide (DMSO). Aliquots were then diluted into ACSF to reach the final concentration. D-AP5 (ref. Ab120003, Abcam, batch: APN11040-2-2) was dissolved as a 30 millimolar stock solution in Milli-Q water, aliquoted and stored at −20° C. until use. Aliquots were thawed and vortexed each day of experiment and then 1000× diluted into ACSF to reach the final concentration of 30 micromolar. NBQX (ref: Ab120045, Abcam, batch: APN10009-4-1) was dissolved as a 10 millimolar stock solution in DMSO (dimethylsulfoxide), aliquoted and stored at −20° C. until use. Aliquots were thawed and vortexed each day of experiment and then 1000× diluted into ACSF to reach the final concentration of 10 micromolar.

All data was recorded with a MEA set-up, commercially available from MultiChannel Systems MCS GmbH (Reutlingen, Germany), and composed of a 4-channel stimulus generator and a 60-channel amplifier head-stage connected to a 60-channels A/D card. Software for stimulation, recordings and analysis were commercially available from Multi Channel Systems: MC Stim (3.2.4 release) and MC Rack (4.0.0 release), respectively. All of the experiments were carried out with 3-dimensional MEA (Ayanda Biosystems, S.A., CH-1015 Lausanne, Switzerland) consisting of 60 tip-shaped and 60-μm-high electrodes spaced by 100 μm. The MEA electrodes were made of platinum with kΩ450<impedance<600 kΩ.

A 350-μm thick mouse hippocampal slice was disposed on the multi-electrode array (100 μm distant electrodes). One electrode was chosen to stimulate Schaeffer collaterals at the CA3/CA1 interface. An I/O curve was performed to monitor evoked-responses for stimulations between 100 and 800 μA (micro-Amperes), by 100 μA steps. The stimulus was a monopolar biphasic current pulse (negative for 60 microseconds and then positive for 60 microseconds), set to evoke 40% of the maximal amplitude response (as determined with the I/O curve) and was applied every 30 seconds to evoke “responses” (i.e., field Excitatory Post Synaptic Potentials; fEPSP) in the CA1 region.

Effect of oleic acid on NMDA dependant LTP induced by a weak tetanus was analyzed. Following a 10-minute period to verify the baseline stability of fEPSP to elicit a weak potentiation, a weak tetanus (10 stimulations at 100 Hz for 0.1 s at 20% of IMAX (Kanno et al., Brain research, 2004)) was used. Weak tetanus-induced potentiation was followed over an approximately 40-minute period.

Evoked-responses (fEPSP) were recorded if they satisfied quality criteria described in Standard Operating Procedures: correct location, stable baseline (fluctuation within +/−10% during ten consecutive minutes), amplitude>100 μV after background noise subtraction. The fEPSP from selected electrodes were simultaneously sampled at 5 kHz and recorded on the hard disk of a PC until offline analysis. In parallel, fEPSP amplitudes of selected electrodes were compiled online (with MC Rack program) to monitor and to follow the performance of the experiments. Data was plotted in a standard spreadsheet file for off-line analysis. Since fEPSPs result from glutamatergic synaptic transmission consecutive to afferent pathway stimulation, at the end of each experiment, 10 μM NBQX were perfused on the slice to validate the glutamatergic nature of synaptic transmission as well as to subtract background noise at individual electrode level. Control LTP were recorded in parallel, with hippocampal slices prepared from the same animals as the ones used to evaluate the compounds.

During the experiments, the slices were continuously perfused with ACSF solutions (bubbled with 95% O₂-5% CO₂) at the rate of 3 milliliters/minute with a peristaltic pump (MEA chamber volume: ˜1 milliliter). Complete solution exchange in the MEA chamber was achieved 20 seconds after the switch of solutions. The perfusion liquid was continuously pre-heated at 37° C. just before reaching the MEA chamber with a heated-perfusion cannula (PH01, MultiChannel Systems, Reutlingen, Germany). The temperature of the MEA chamber was maintained at 37° C.+/−0.1° C. with a Peltier element located in the MEA amplifier headstage.

I/O curves from identical experimental conditions were analysed using repeated measure two-way ANOVA followed by a post hoc Bonferroni test. LTP from identical experimental conditions were analysed using one-way ANOVA followed by a post hoc Dunnett test. Statistical analysis was performed by using Prism 5.0 software. A critical P value of P<0.05 was considered significant for the statistical tests used throughout the study.

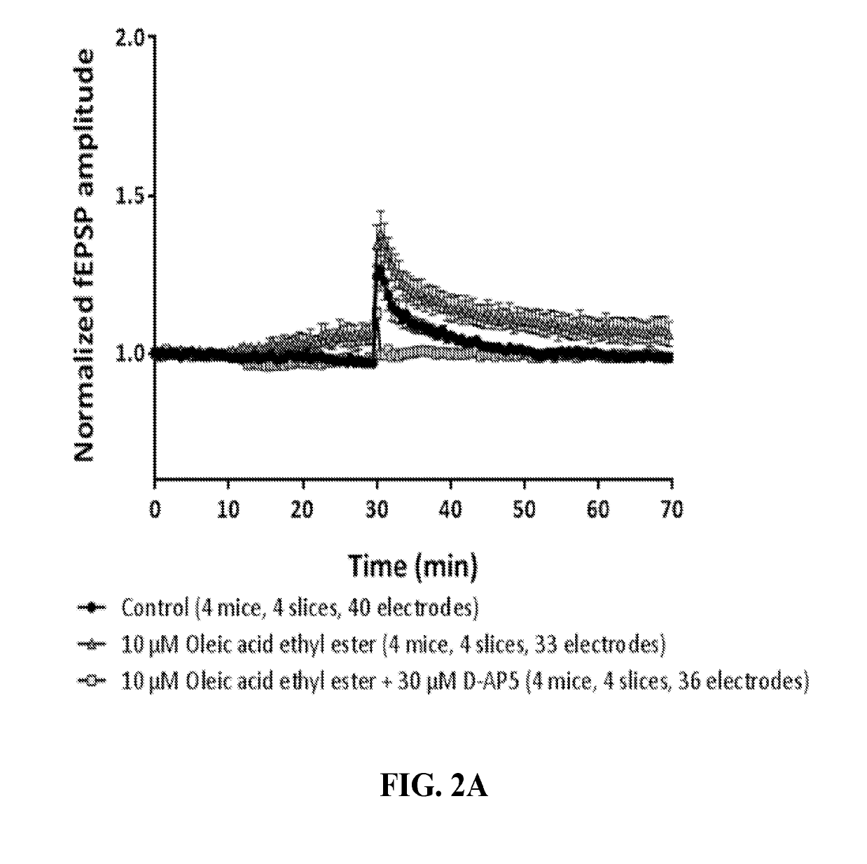

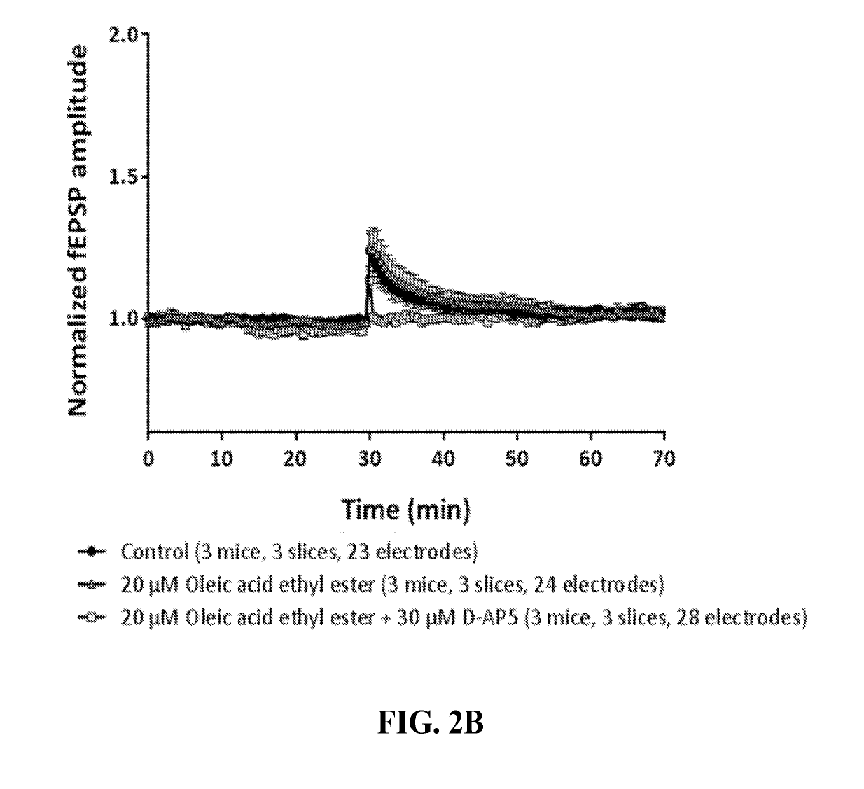

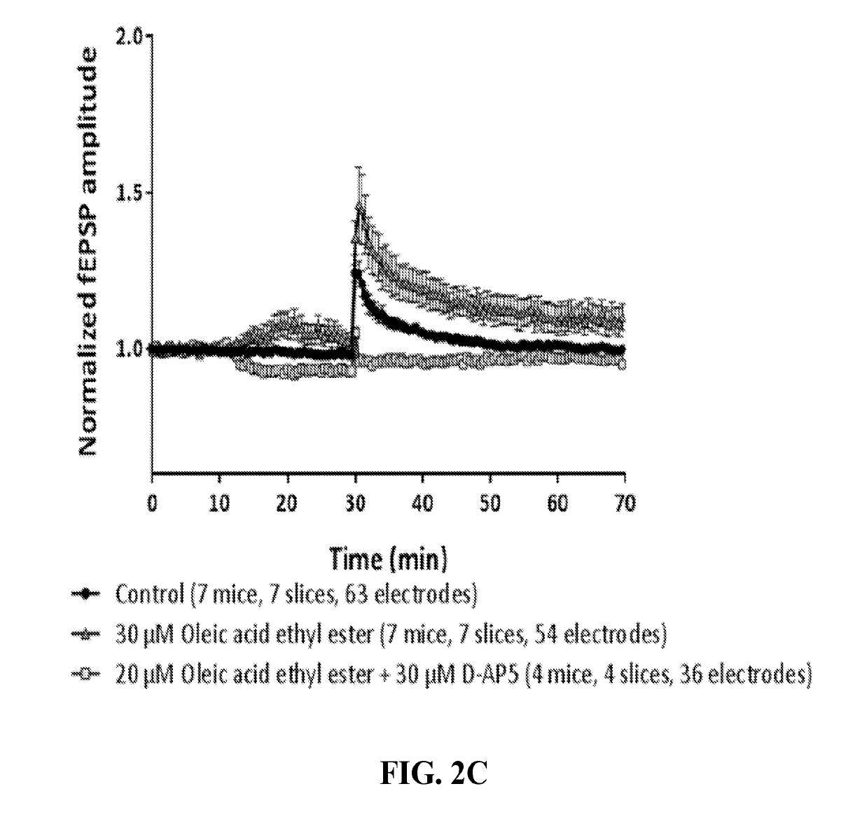

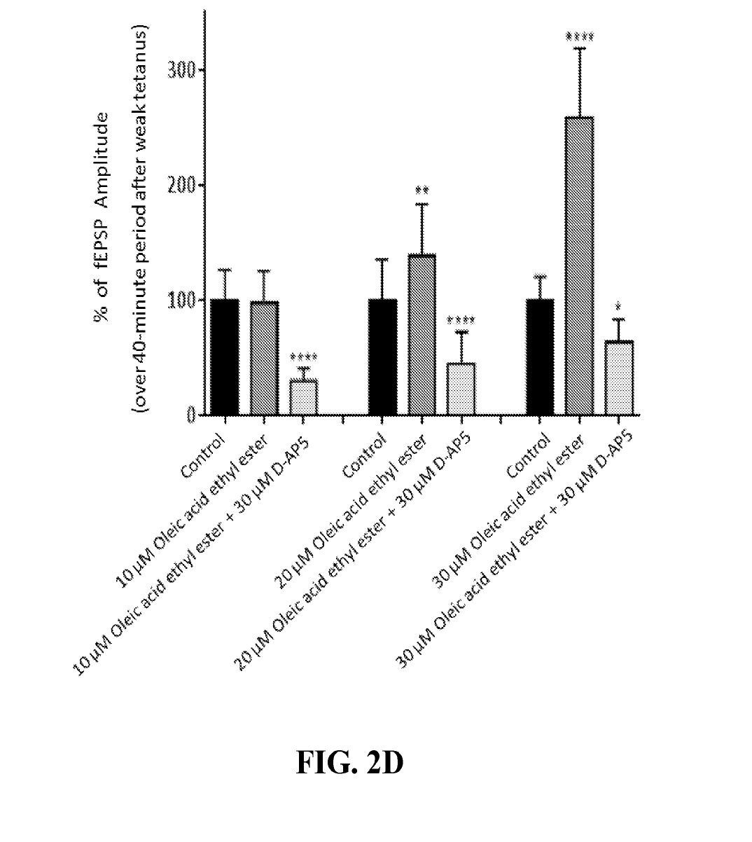

As shown in FIGS. 2A-2D, oleic acid (ethyl ester) at 20 and 30 μM significantly enhanced NMDAR dependant hippocampal LTP induced by weak tetanic stimulation (10 stimulations applied at 100 Hz for 0.1 seconds). At 10 μM Oleic acid did not produce any effect on NMDAR dependant hippocampal LTP. Weak stimulation resulted in weak potentiation such as that which normally occurs during short term memory formation.

### Example 6

In this Example, the administration of oleic acid (ethyl ester) and choline chloride combination on NMDAR dependent hippocampal LTP in mice is analyzed. As used herein the term AMPA is an acronym for α-amino-3-hydroxy-5-methyl-4-isoxazolepropionic acid; the terms AMPA receptor and AMPAR are used interchangeably. As used herein the term NDMA is an acronym for N-Methyl-D-aspartic acid or N-Methyl-D-aspartate; the terms NDMA receptor and NDMAR are used interchangeably.

Long lasting changes in the strength of AMPA and NMDA receptor mediated glutamatergic synaptic transmission in the hippocampus, known as hippocampal synaptic plasticity, is a key underlying molecular mechanism in learning and memory. AMPA and NMDA dependant hippocampal synaptic plasticity is associated with trafficking and delivery of AMPA receptors at the synapse in response to NMDAR activation. Previous studies have shown that in cognitive decline associated with aging or Alzheimer's disease onset, the strength of AMPA and NMDA synaptic transmission is compromised due to the internalization of NMDA and AMPA receptors, leading to deficits in NMDAR and AMPAR expression at the synapse. During learning, neuronal activation leads to pre-synaptic release of glutamate resulting in the activation of post-synaptic AMPA and NMDA receptors. Activation of post-synaptic AMPA receptors results in the flow of Na⁺ ions, causing the depolarization of post-synaptic neuron. This depolarization abolishes the blocking of NMDA receptors by Mg²⁺, resulting in the opening of NMDA receptor channels for Na⁺ and Ca²⁺ ions. Ca²⁺ influx through NMDA receptors is known to trigger intracellular signaling cascades, notably the phosphorylation of Ca²⁺/calmodulin-dependent protein kinase (CaMKII) that phosphorylates AMPA receptors and modulates the channel properties, ultimately resulting in long-term potentiation (LTP), the physiological correlate of synaptic plasticity (learning and memory).

Therefore in this Example, NMDAR dependant hippocampal LTP is used as a physiological marker to assess the efficacy of the combination of oleic acid and choline in improving or enhancing cognitive function. To evaluate the efficacy of the combination of oleic acid and choline in improving or enhancing cognitive function, NMDAR dependant LTP is recorded on hippocampal brain slices in vitro.

Experiments are carried out with 7-9 weeks-old C57/Black6 mice (from Elevage Janvier, Le Genest St Isle, France). Animals are housed and used in accordance with the French and European legislations for animal care. The mice are sacrificed by fast decapitation, without previous anaesthesia. The brain is quickly removed and soaked in ice-cold oxygenated buffer having the following composition:

| Cutting Solution | Final concentration (mM) |
|---|---|
| KCl | 2 |
| NaH₂PO₄ | 1.2 |
| MgCl₂ | 7 |
| CaCl₂ | 0.5 |
| NaHCO₃ | 26 |
| D-glucose | 11 |
| Saccharose | 250 |

Hippocampus slices (350 micrometer) are cut with a MacIlwain tissue-chopper and incubated at room temperature for at least 60 minutes in Artificial Cerebro-Spinal Fluid (ACSF) having the following composition:

| ACSF | Final concentration (mM) |
|---|---|
| NaCl | 126 |
| KCl | 3.5 |
| NaH₂PO₄ | 1.2 |
| MgCl₂ | 1.3 |
| CaCl₂ | 2 |
| NaHCO₃ | 25 |
| D-glucose | 11 |

During experiments, slices are continuously perfused with oxygenated ACSF.

Choline chloride is dissolved in Milli-Q water 100 millimolar stock solution in Milli-Q water and stored at −20° C. until use. Oleic acid ethyl ester is dissolved in DMSO. Aliquots of choline chloride are thawed and vortexed each day of experiment. Oleic acid ethyl ester is freshly prepared on the day of experiment. Both oleic acid and choline chloride are diluted into ACSF to reach the final concentration. D-AP5 (ref. Ab120003, Abcam, batch: APN11040-2-2) is dissolved as a 30 millimolar stock solution in Milli-Q water, aliquoted and stored at −20° C. until use. Aliquots are thawed and vortexed each day of experiment and then 1000× diluted into ACSF to reach the final concentration of 30 micromolar. NBQX (ref: Ab120045, Abcam, batch: APN10009-4-1) is dissolved as a 10 millimolar stock solution in DMSO (dimethylsulfoxide), aliquoted and stored at −20° C. until use. Aliquots are thawed and vortexed each day of experiment and then 1000× diluted into ACSF to reach the final concentration of 10 micromolar.

All data is recorded with a MEA set-up, commercially available from MultiChannel Systems MCS GmbH (Reutlingen, Germany), and composed of a 4-channel stimulus generator and a 60-channel amplifier head-stage connected to a 60-channels A/D card. Software for stimulation, recordings and analysis were commercially available from Multi Channel Systems: MC Stim (3.2.4 release) and MC Rack (4.0.0 release), respectively. All of the experiments are carried out with 3-dimensional MEA (Ayanda Biosystems, S.A., CH-1015 Lausanne, Switzerland) consisting of 60 tip-shaped and 60-μm-high electrodes spaced by 100 μm. The MEA electrodes are made of platinum with kΩ450<impedance<600 kΩ.

A 350-μm thick mouse hippocampal slice is disposed on the multi-electrode array (100 μm distant electrodes). One electrode is chosen to stimulate Schaeffer collaterals at the CA3/CA1 interface. An I/O curve is performed to monitor evoked-responses for stimulations between 100 and 800 μA (micro-Amperes), by 100 μA steps. The stimulus is a monopolar biphasic current pulse (negative for 60 microseconds and then positive for 60 microseconds), set to evoke 40% of the maximal amplitude response (as determined with the I/O curve) and was applied every 30 seconds to evoke “responses” (i.e. field Excitatory Post Synaptic Potentials; fEPSP) in the CA1 region.

The effect of oleic acid and choline chloride combination on NMDA dependant LTP induced by a weak tetanus is analyzed. Following a 10-minute period to verify the baseline stability of fEPSP to elicit a weak potentiation, a weak tetanus (10 stimulations at 100 Hz for 0.1 s at 20% of IMAX (Kanno et al., Brain research, 2004)) is used. Weak tetanus-induced potentiation is followed over an approximately 40-minute period.

Evoked-responses (fEPSP) are recorded if they satisfy quality criteria described in Standard Operating Procedures: correct location, stable baseline (fluctuation within +/−10% during ten consecutive minutes), amplitude>100 μV after background noise subtraction. The fEPSP from selected electrodes are simultaneously sampled at 5 kHz and recorded on the hard disk of a PC until offline analysis. In parallel, fEPSP amplitudes of selected electrodes are compiled online (with MC Rack program) to monitor and to follow the performance of the experiments. Data is plotted in a standard spreadsheet file for off-line analysis. Since fEPSPs result from glutamatergic synaptic transmission consecutive to afferent pathway stimulation, at the end of each experiment, 10 μM NBQX are perfused on the slice to validate the glutamatergic nature of synaptic transmission as well as to subtract background noise at individual electrode level. Control LTP are recorded in parallel, with hippocampal slices prepared from the same animals as the ones used to evaluate the compounds.

During the experiments, the slices are continuously perfused with ACSF solutions (bubbled with 95% O₂-5% CO₂) at the rate of 3 milliliters/minute with a peristaltic pump (MEA chamber volume: ˜1 milliliter). Complete solution exchange in the MEA chamber is achieved 20 seconds after the switch of solutions. The perfusion liquid is continuously pre-heated at 37° C. just before reaching the MEA chamber with a heated-perfusion cannula (PH01, MultiChannel Systems, Reutlingen, Germany). The temperature of the MEA chamber is maintained at 37° C.+/−0.1° C. with a Peltier element located in the MEA amplifier headstage.

I/O curves from identical experimental conditions are analysed using repeated measure two-way ANOVA followed by a post hoc Bonferroni test. LTP from identical experimental conditions are analysed using one-way ANOVA followed by a post hoc Dunnett test. Statistical analysis is performed by using Prism 5.0 software. A critical P value of P<0.05 is considered significant for the statistical tests used throughout the study.

### Example 7

In this Example, the administration of oleic acid (ethyl ester), choline chloride and soy-PS combination on NMDAR dependent hippocampal LTP in mice is analyzed. As used herein the term AMPA is an acronym for α-amino-3-hydroxy-5-methyl-4-isoxazolepropionic acid; the terms AMPA receptor and AMPAR are used interchangeably. As used herein the term NDMA is an acronym for N-Methyl-D-aspartic acid or N-Methyl-D-aspartate; the terms NDMA receptor and NDMAR are used interchangeably.

Long lasting changes in the strength of AMPA and NMDA receptor mediated glutamatergic synaptic transmission in the hippocampus, known as hippocampal synaptic plasticity, is a key underlying molecular mechanism in learning and memory. AMPA and NMDA dependant hippocampal synaptic plasticity is associated with trafficking and delivery of AMPA receptors at the synapse in response to NMDAR activation. Previous studies have shown that in cognitive decline associated with aging or Alzheimer's disease onset, the strength of AMPA and NMDA synaptic transmission is compromised due to the internalization of NMDA and AMPA receptors, leading to deficits in NMDAR and AMPAR expression at the synapse. During learning, neuronal activation leads to pre-synaptic release of glutamate resulting in the activation of post-synaptic AMPA and NMDA receptors. Activation of post-synaptic AMPA receptors results in the flow of Na⁺ ions, causing the depolarization of post-synaptic neuron. This depolarization abolishes the blocking of NMDA receptors by Mg²⁺, resulting in the opening of NMDA receptor channels for Na⁺ and Ca²⁺ ions. Ca²⁺ influx through NMDA receptors is known to trigger intracellular signaling cascades, notably the phosphorylation of Ca²⁺/calmodulin-dependent protein kinase (CaMKII) that phosphorylates AMPA receptors and modulates the channel properties, ultimately resulting in long-term potentiation (LTP), the physiological correlate of synaptic plasticity (learning and memory).

Therefore in this Example, NMDAR dependant hippocampal LTP is used as a physiological marker to assess the efficacy of the combination of oleic acid, choline chloride and soy-PS in improving or enhancing cognitive function. To evaluate the efficacy of the combination of oleic acid, choline chloride and soy-PS in improving or enhancing cognitive function, NMDAR dependant LTP is recorded on hippocampal brain slices in vitro.

Experiments are carried out with 7-9 weeks-old C57/Black6 mice (from Elevage Janvier, Le Genest St Isle, France). Animals are housed and used in accordance with the French and European legislations for animal care. The mice are sacrificed by fast decapitation, without previous anaesthesia. The brains are quickly removed and soaked in ice-cold oxygenated buffer having the following composition:

| Cutting Solution | Final concentration (mM) |
|---|---|
| KCl | 2 |
| NaH₂PO₄ | 1.2 |
| MgCl₂ | 7 |
| CaCl₂ | 0.5 |
| NaHCO₃ | 26 |
| D-glucose | 11 |
| Saccharose | 250 |

Hippocampus slices (350 micrometer) are cut with a MacIlwain tissue-chopper and incubated at room temperature for at least 60 minutes in Artificial Cerebro-Spinal Fluid (ACSF) having the following composition:

| ACSF | Final concentration (mM) |
|---|---|
| NaCl | 126 |
| KCl | 3.5 |
| NaH₂PO₄ | 1.2 |
| MgCl₂ | 1.3 |
| CaCl₂ | 2 |
| NaHCO₃ | 25 |
| D-glucose | 11 |

During experiments, slices are continuously perfused with oxygenated ACSF. Choline Chloride is dissolved in Milli-Q water 100 millimolar stock solution in Milli-Q water and stored at −20° C. until use. Oleic acid ethyl ester is dissolved in DMSO. Aliquots of choline chloride are thawed and vortexed each day of experiment. Oleic acid ethyl ester is freshly prepared on the day of experiment. Both oleic acid and choline chloride are diluted into ACSF to reach the final concentration. Soy-PS liposomes are prepared with 1,2-dioleoyl-sn-glycero-3-phosphocholine (DOPC) as a 63.4 mM stock solution. Soy-PS is directly dissolved into ACSF to reach its final concentration (2.5, 5, 10 and 30 μM). Control DOPC liposomes solution (sent by Avanti Polar Lipids, Inc.,) is a 5.4 mM stock solution. As the Soy-PS:DOPC ratio is 9:1, the concentration of DOPC is adjusted to 10 μM final (based on the probable highest Soy-PS concentration to be tested). D-AP5 (ref. Ab120003, Abcam, batch: APN11040-2-2) is dissolved as a 30 millimolar stock solution in Milli-Q water, aliquoted and stored at −20° C. until use. Aliquots are thawed and vortexed each day of experiment and then 1000× diluted into ACSF to reach the final concentration of 30 micromolar. NBQX (ref: Ab120045, Abcam, batch: APN10009-4-1) is dissolved as a 10 millimolar stock solution in DMSO (dimethylsulfoxide), aliquoted and stored at −20° C. until use. Aliquots were thawed and vortexed each day of experiment and then 1000× diluted into ACSF to reach the final concentration of 10 micromolar.

All data is recorded with a MEA set-up, commercially available from MultiChannel Systems MCS GmbH (Reutlingen, Germany), and composed of a 4-channel stimulus generator and a 60-channel amplifier head-stage connected to a 60-channels A/D card. Software for stimulation, recordings and analysis were commercially available from Multi Channel Systems: MC Stim (3.2.4 release) and MC Rack (4.0.0 release), respectively. All of the experiments are carried out with 3-dimensional MEA (Ayanda Biosystems, S.A., CH-1015 Lausanne, Switzerland) consisting of 60 tip-shaped and 60-μm-high electrodes spaced by 100 μm. The MEA electrodes are made of platinum with kΩ450<impedance<600 kΩ.

A 350-μm thick mouse hippocampal slice is disposed on the multi-electrode array (100 μm distant electrodes). One electrode is chosen to stimulate Schaeffer collaterals at the CA3/CA1 interface. An I/O curve is performed to monitor evoked-responses for stimulations between 100 and 800 μA (micro-Amperes), by 100 μA steps. The stimulus is a monopolar biphasic current pulse (negative for 60 microseconds and then positive for 60 microseconds), set to evoke 40% of the maximal amplitude response (as determined with the I/O curve) and is applied every 30 seconds to evoke “responses” (i.e. field Excitatory Post Synaptic Potentials; fEPSP) in the CA1 region.

The effect of the combination of oleic acid, choline chloride, and soy-PS on NMDA dependant LTP induced by a weak tetanus is analyzed. Following a 10-minute period to verify the baseline stability of fEPSP to elicit a weak potentiation, a weak tetanus (10 stimulations at 100 Hz for 0.1 s at 20% of IMAX (Kanno et al., Brain research, 2004)) is used. Weak tetanus-induced potentiation is followed over an approximately 40-minute period.

Evoked-responses (fEPSP) are recorded if they satisfy quality criteria described in Standard Operating Procedures: correct location, stable baseline (fluctuation within +/−10% during ten consecutive minutes), amplitude>100 μV after background noise subtraction. The fEPSP from selected electrodes are simultaneously sampled at 5 kHz and recorded on the hard disk of a PC until offline analysis. In parallel, fEPSP amplitudes of selected electrodes are compiled online (with MC Rack program) to monitor and to follow the performance of the experiments. Data is plotted in a standard spreadsheet file for off-line analysis. Since fEPSPs result from glutamatergic synaptic transmission consecutive to afferent pathway stimulation, at the end of each experiment, 10 μM NBQX are perfused on the slice to validate the glutamatergic nature of synaptic transmission as well as to subtract background noise at individual electrode level. Control LTP are recorded in parallel, with hippocampal slices prepared from the same animals as the ones used to evaluate the compounds.

During the experiments, the slices are continuously perfused with ACSF solutions (bubbled with 95% O₂-5% CO₂) at the rate of 3 milliliters/minute with a peristaltic pump (MEA chamber volume: ˜1 milliliter). Complete solution exchange in the MEA chamber is achieved 20 seconds after the switch of solutions. The perfusion liquid is continuously pre-heated at 37° C. just before reaching the MEA chamber with a heated-perfusion cannula (PH01, MultiChannel Systems, Reutlingen, Germany). The temperature of the MEA chamber is maintained at 37° C.+/−0.1° C. with a Peltier element located in the MEA amplifier headstage.

I/O curves from identical experimental conditions are analysed using repeated measure two-way ANOVA followed by a post hoc Bonferroni test. LTP from identical experimental conditions are analysed using one-way ANOVA followed by a post hoc Dunnett test. Statistical analysis is performed by using Prism 5.0 software. A critical P value of P<0.05 is considered significant for the statistical tests used throughout the study.

### Example 8

In this example, the efficacy of different doses of PS (soy), choline and oleic acid alone and/or in combination in enhancing cognitive functioning in animal model is analyzed. The efficacy of PS, choline and oleic acid on cognitive functioning is measured based on their effect on acetylcholine levels as well as on Long term potentiation (LTP) in vivo (in an animal model). For example, doses such as oleic acid at 1 g/Kg body weight, PS (soy) at 10 mg/Kg body weight, and choline at 15 mg/Kg body weight are used alone or in combination to determine the potential synergistic or additive effects or complimentary mode of action The acetylcholine (ACh) depletion and the resulting hypofunction of the cholinergic neuronal system is one of the major causes of cognitive dysfunctioning.

Long lasting changes in the strength of AMPA and NMDA receptor mediated glutamatergic synaptic transmission in the hippocampus, known as hippocampal synaptic plasticity, is a key underlying molecular mechanism in learning and memory. AMPA and NMDA dependant hippocampal synaptic plasticity is associated with trafficking and delivery of AMPA receptors at the synapse in response to NMDAR activation. Previous studies have shown that in cognitive decline associated with aging or Alzheimer's disease onset, the strength of AMPA and NMDA synaptic transmission is compromised due to the internalization of NMDA and AMPA receptors, leading to deficits in NMDAR and AMPAR expression at the synapse. During learning, neuronal activation leads to pre-synaptic release of glutamate resulting in the activation of post-synaptic AMPA and NMDA receptors. Activation of post-synaptic AMPA receptors results in the flow of Na⁺ ions, causing the depolarization of post-synaptic neuron. This depolarization abolishes the blocking of NMDA receptors by Mg²⁺, resulting in the opening of NMDA receptor channels for Na⁺ and Ca²⁺ ions. Ca²⁺ influx through NMDA receptors is known to trigger intracellular signaling cascades, notably the phosphorylation of Ca²⁺/calmodulin-dependent protein kinase (CaMKII) that phosphorylates AMPA receptors and modulates the channel properties, ultimately resulting in long-term potentiation (LTP), the physiological correlate of synaptic plasticity (learning and memory).

In this Example, hippocampal LTP is used as a physiological marker to assess the efficacy of PS (soy), choline and oleic acid in improving or enhancing cognitive function. To evaluate the efficacy of PS (soy), choline and oleic acid in improving or enhancing cognitive function, hippocampal LTP is recorded in vivo.

In this Example, the administration of oleic acid (ethyl ester), choline chloride and PS (soy) in combination on hippocampal LTP in vivo is analyzed in a rodent model (rats). As used herein the term AMPA is an acronym for α-amino-3-hydroxy-5-methyl-4-isoxazolepropionic acid; the terms AMPA receptor and AMPAR are used interchangeably. As used herein the term NDMA is an acronym for N-Methyl-D-aspartic acid or N-Methyl-D-aspartate; the terms NDMA receptor and NDMAR are used interchangeably.

Male adult Sprague Dawley rats (280-350 g; Harlan or Charles River) are used for the experiments. Before surgery the animals are group housed (in groups of five) in plastic cages and have access to food and water ad libitum. The animals are placed on a special diet (Teklad Global 18% Protein Rodent Diet, 2018S) for three weeks before surgery. There is an automatic control of light cycle, temperature and humidity. Light hours are 07:00-19:00. The temperature and humidity is continually monitored so that the values remain within the target ranges of 22° C.±2° C. and 55%±15%, respectively. When necessary, the rats are anesthetized using isoflurane 2% and O₂. Before the surgery, Fynadine (1 mg/kg s.c.) is administered for analgesia during surgery and recovery. A mixture of bupivacaine and adrenaline is used for local anesthesia at the site of incision.

Each animal is placed into a stereotaxic frame (Kopf Instruments, USA). One guide cannula is inserted into both brain hemispheres according to the Paxinos and Watson (1982) brain atlas. Each guide fits a I-shaped probe with 4 mm exposed surface of hospal membrane (Brainlink, the Netherlands). The guide is placed in order to position the tip of the probe at the following coordinates: AP −5.3 mm (to bregma), lateral +/−4.8 mm (to midline), ventral −8.0 mm (to dura) with toothbar set at −3.3 mm, corresponding to the ventral hippocampus. The guide is fixed to the skull with dental cement and a stainless steel screw. After surgery animals are housed individually or two animals per cage; food and water are available ad libitum. The animals receive the compound(s) and/or a combination of compounds (i.e., soy-PS, choline and oleic acid) acutely and during 6 weeks by oral gavage. During the microdialysis experiments (on day 1 and 42) the animals also receive compound and/or a combination of compounds.

PS (soy), choline and oleic acid alone or in combination are administered at least 4 days after surgery. For example, doses such as oleic acid at 1 g/Kg body weight, PS (soy) at 10 mg/Kg body weight, and choline at 15 mg/Kg body weight are used. On the day of the experiment, the probes in the rats are connected with flexible PEEK tubing to a microperfusion pump (Syringe pump UV 8301501, TSE, Germany) and perfused with artificial cerebrospinal fluid (perfusate), containing 147 mM NaCl, 3.0 mM KCl, 1.2 mM CaCl₂, and 1.2 mM MgCl₂ with a flow rate of 1.5 μl/minute. After 2 hours pre-stabilization, microdialysis samples are collected off-line for 20 minute periods into mini-vials, already containing 10 μL of 0.02 M formic/ascorbic acid, by an automated fraction collector (CMA 142, Sweden). Three samples are collected for basal concentration determination and 12 samples are collected after a challenge with compound. After collection, samples are stored at −80° C. for acetylcholine analysis, as mentioned below. After the chronic experiment, the rats are sacrificed and the brain is removed and stored at −80° C. for later use.

Concentrations of the neurotransmitters are determined by HPLC with tandem mass spectrometry (MS/MS) detection. MS analyses are performed using a API 3000, 4000 or 5000 MS/MS system consisting of a API 3000, 4000 or 5000 MS/MS detector and a Turbo Ion Spray interface (both from Applied Biosystems, the Netherlands). Data is calibrated and quantified using the Analyst™ data system (Applied Biosystem, version 1.4.2/1.5.1).

For the in vivo LTP study, Male adult Sprague Dawley rats (280-350 grams; from Harlan or Charles River) are used for the experiments. Before surgery, the animals are group housed (in groups of five) in plastic cages and have access to food and water ad libitum. There is an automatic control of light cycle, temperature and humidity. Light hours are 07:00-19:00. The temperature and humidity are continually monitored so that the values remain within the target ranges of 22° C.±2° C. and 55%±15% respectively. Experiments are conducted in accordance with the declarations of Helsinki and are approved by the Institutional Animal Care and Use Committee of the University of Groningen.

Experimental procedures are carried out under deep urethane anesthesia (1-2 g/kg, i.p. eye-ointment is applied). The skin over the skull is opened by incision and local analgesic (bupivacaine/adrenaline) are applied to the periost. A small hole is drilled over the area of interest, based on the coordinates according to Paxinos and Watson. The anesthetic depth is checked by response to toe-pinches. Surgery and experiment are performed on a heating pad, in order to maintain normal body temperature of the rat. During the experiment, s.c. saline is administered to prevent dehydration.

A recording electrode is inserted into the designated brain area (e.g., CA1 of hippocampus), and bipolar coated stainless stimulating electrode-to the area of afferent fibers innervating this brain area (e.g., Schaffer Collateral). Upon establishment of satisfactory and classical EPSP using electrophysiological criteria, stabilization is permitted for about 30 minutes. After verification of the correct area, an input/output curve is generated by applying stimulation of varying intensities and measuring the output amplitudes of the EPSP. A brief stabilization period of about 30 minutes takes place and at the end of this period the I/O curve is repeated in order to check that the I/O curve is accurate. When satisfactory accuracy is reached the electrode is glued into place with Superglue to provide additional stability and thereafter fixed in place with dental cement. The dental cement serves to provide stability and also to reduce electrical noise. Test EPSPs to be evoked at a frequency of 0.033 Hz (voltage according to input/output (I/O) curve previously established—given at 30 second intervals while looking for EPSPs; and potentially while recording following the trains. For studies, EPSP amplitudes of 50% of maximal is used for the continual test pulses. LTP is induced with two-theta-burst protocols (burst 1 and 2): 4 trains each of pulses delivered at 75 Hz with an interburst-interval of 200 ms. During the theta-bursts the intensity is increased to elicit 75% maximal EPSP, or similar. During recording, reducing stimulation at 50% of maximal EPSP (from the I/O curve) is used.

The complete disclosure of all patents, patent applications, and publications, and electronically available material cited herein are incorporated by reference. While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus, and illustrative examples shown and described. Accordingly, departures may be made from such details without departing from the spirit or scope of the applicant's general inventive concept.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components. While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus, and illustrative examples shown and described. Accordingly, departures may be made from such details without departing from the spirit or scope of the applicant's general inventive concept.

1. A method of improving cognitive performance, comprising orally administering a composition comprising an effective amount of each of phosphatidyl serine, choline, and oleic acid to a subject.

2. The method of claim 1, wherein the subject is a human.

3. The method of claim 1, wherein the composition is a nutritional composition.

4. The method of claim 3, wherein the nutritional composition contains 150-500 calories per serving and is in the form of a powder suitable for reconstitution to a liquid, a liquid or a bar.

5. The method of claim 3, wherein the nutritional composition further comprises at least one source of carbohydrate and at least one source of protein.

6. (canceled)

7. The method of claim 5, wherein the carbohydrate comprises sucrose and the protein comprises casein.

8. The method of claim 1, wherein the amount of phosphatidyl serine ranges from 20 mg/day to 600 mg/day.

9. The method of claim 1, wherein the amount of choline ranges from 20 mg/day to 600 mg/day.

10. (canceled)

11. The method of claim 1, wherein the amount of oleic acid ranges from 0.1 g/day to 30 g/day.

12. The method of claim 1, wherein the amount of phosphatidyl serine ranges from 20 mg/day to 600 mg/day, the amount of choline ranges from 20 mg/day to 600 mg/day, and the amount of oleic acid ranges from 0.1 g/day to 30 g/day.

13. (canceled)

14. The method of claim 1, wherein the amount of phosphatidyl serine ranges from 50 mg/day to 300 mg/day, the amount of choline ranges from 50 mg/day to 550 mg/day, and the amount of oleic acid ranges from 0.5 g/day to 25 g/day.

15. The method of claim 1, wherein the composition is administered to the subject every day for at least two weeks.

16. The method of claim 1, wherein the cognitive performance that is improved is memory.

17. The method of claim 1, wherein the cognitive performance that is improved is intelligence.

18. The method of claim 2, wherein the human is an elderly human.

19. The method of claim 18, wherein the elderly human has been diagnosed with dementia or cognitive dysfunction.

20.-29. (canceled)

30. A nutritional composition comprising phosphatidyl serine, choline, oleic acid, at least one source of carbohydrate, and at least one source of protein.

31. The nutritional composition of claim 30, wherein the composition further comprises at least one source of fat.

32. The nutritional composition of claim 30, wherein the composition provides 150-500 calories per serving and is in the form of a powder suitable for reconstitution to a liquid, a liquid or a bar.

33. (canceled)

34. (canceled)

35. The nutritional composition of claim 30, wherein the composition comprises from 10 mg to 300 mg of phosphatidyl serine per serving.

36. The nutritional composition of claim 30, wherein the composition comprises from 10 mg to 300 mg of choline per serving.

37. (canceled)

38. The nutritional composition of claim 30, wherein the composition comprises from 0.1 grams to 15 grams of oleic acid per serving.

39. (canceled)

40. The nutritional composition of claim 30, wherein the composition comprises from 10 mg to 300 mg of phosphatidyl serine, from 10 mg to 300 mg of choline, and from 0.1 grams to 15 grams of oleic acid per serving.

41. (canceled)
